(19) United States
(12) Patent Application Publication
(10) Pub. No.: US 2023/0272362 A1
(43) Pub. Date: Aug. 31, 2023

(54) ENZYME FOR DECOMPOSING ACRYLAMIDE

(71) Applicant: ANKA ANGEWANDTE KAFFEETECHNOLOGIE GMBH, Bremen (DE)

(72) Inventors: Oliver SÜSSE-HERRMANN, Hude (DE); Sabrina KÖPKE, Leipzig (DE); Andreas VOGEL, Leipzig (DE); Claudia FELLER, Leipzig (DE); Sebastian BARTSCH, Leipzig (DE)

(21) Appl. No.: 17/794,358

(22) PCT Filed: Jan. 21, 2021
(86) PCT No.: PCT/EP2021/051283

§ 371 Date: Jul. 21, 2022

(30) Foreign Application Priority Data
Jan. 21, 2020 (EP) 20152853.6

(51) Int. Cl. C12N 9/80; A23F 5/16; A23L 5/20

(52) U.S. Cl. CPC C12N 9/80; A23F 5/163; A23L 5/25; C12Y 305/01004

(57) ABSTRACT

The invention is mainly in the field of the production of food and luxury foods, for example coffee and coffee substitute products. Enzymes are provided which are capable of degrading acrylamide - preferably also at temperatures above 50° C., in particular in temperature ranges which occur in the production of coffee/coffee substitute products, and/or at a pH value between pH 4 and pH 7, as is usual in the production of coffee/coffee substitute products. Furthermore, methods for degrading acrylamide from preparations selected from semi-finished goods as well as finished goods are provided. Also, the present invention relates to preparations having a reduced acrylamide content compared to preparations which have not been subjected to the method for removing acrylamide according to the invention by means of the enzymes according to the invention.

The invention is in the field of the production of food and luxury food, especially coffee and coffee substitute products. New enzymes are provided that are capable of degrading acrylamide - preferably at temperatures above 50° C., in particular in temperature ranges that occur in the production of coffee/coffee substitute products, and/or at a pH value between pH 4 and pH 7, as is common in the production of coffee/coffee substitute products. Furthermore, methods for degrading acrylamide from preparations selected from semi-finished goods as well as finished goods are provided. Also, the present invention relates to preparations having a reduced acrylamide content compared to preparations which have not been subjected to the process for removing acrylamide according to the invention by means of the enzymes according to the invention.

The demands of end consumers for food and luxury food that are consistently safe and safe to consume are continuously high and place special requirements on producers. A new EU regulation from 2018 (EU 2017/2158) classifies acrylamide as a process contaminant and potential health risk for end consumers. Accordingly, food and luxury food producers are obliged to keep the acrylamide content in food and luxury food below a certain level or to reduce it. In animal studies, acrylamide is carcinogenic and mutagenic. Acrylamide is formed in all frying, baking and deep-frying processes of raw materials containing starch and is the product of the heating of asparagine with reducing sugars (e.g. glucose and fructose) as part of the so-called Maillard reaction.

In the particular case of coffee and coffee substitute product production, these processes involve the roasting or extraction of roasted and ground coffee beans and their substitutes, such as chicory, barley or rye. During roasting, coffee beans are typically subjected to temperatures ranging from 145° C. to 250° C., with complex chemical reactions, the Maillard reaction, caramelization and pyrolysis ocurring. These reactions change the chemical, physical and sensory properties of the roasted products, which are elementally important for the taste of the beverage. In addition, other substances are formed that are important for the final product, such as antioxidants (Jin et al. “Relationship between antioxidants and acrylamide formation: A review,” Food Research International, 2013, p. 611-620). Acrylamide is also formed as an undesirable process contaminant by roasting coffee and coffee substitutes (Anese, M. “Acrylamide in Coffee and Coffee Substitutes, Acrylamide in Food, 2016, p.181 - 195). The guideline values for acrylamide in the new EU regulation are 400 µg/kg for roasted coffee, 850 µg/kg for soluble coffee, 500 µg/kg for coffee substitute products made exclusively from cereals, and 4000 µg/kg for products made from chicory.

Acrylamide is also formed in a large number of (other) processes in the food and food luxury industry. For example, acrylamide is formed when potatoes are deep-fried. It is advantageous or even necessary to partially or completely remove acrylamide from semi-finished or finished products, in particular to meet the requirements of the EU Regulation (EU 2017/2158) on the one hand and to obtain a safe end product that is harmless to consumers on the other hand.

Although there are a large number of attempts to reduce the acrylamide content in food and luxury foods, these are not able to remove acrylamide almost completely or completely and gently at economically justifiable costs. For example, in patent application EP 3254568 A1, the acrylamide content is lowered after coffee extraction by using a cationic resin to absorb the acrylamide. This process requires an additional process step and is time-consuming because absorption is a kinetically slow step. Furthermore, only 50% of the acrylamide could be removed, and the cationic resin is an additional cost in the process.

Another way to reduce acrylamide is to reduce the precursors. This is the subject of patent application WO 2013/005145 A1. It discloses a process for the reduction of asparagine and aspartic acid, which starts before the roasting process. Reducing the asparagine content before roasting reduces the acrylamide content of the final product, but the flavor profile of the final product is modified. Furthermore, this process is costly for the user, as additional new equipment is required to perform this process.

Another approach is taken by the authors of patent EP 1745702 B1, which also deals with the enzyme-assisted production of coffee. Here, the starch of the coffee beans is enzymatically degraded and already broken down into its individual building blocks in order to obtain an improved flavor profile in the end product. In this process, the high availability of monosaccharides results in more acrylamide than in standard coffee products. This clearly shows that the right timing of an enzyme treatment is crucial for reducing the acrylamide content.

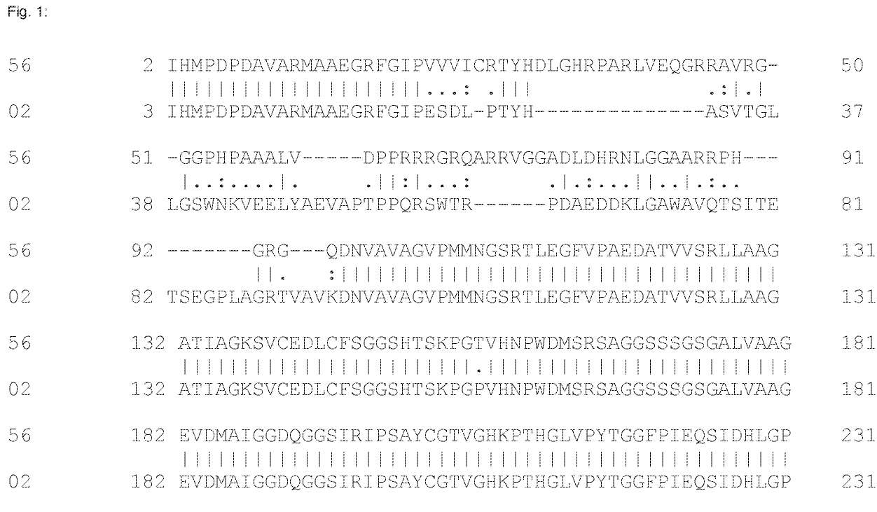

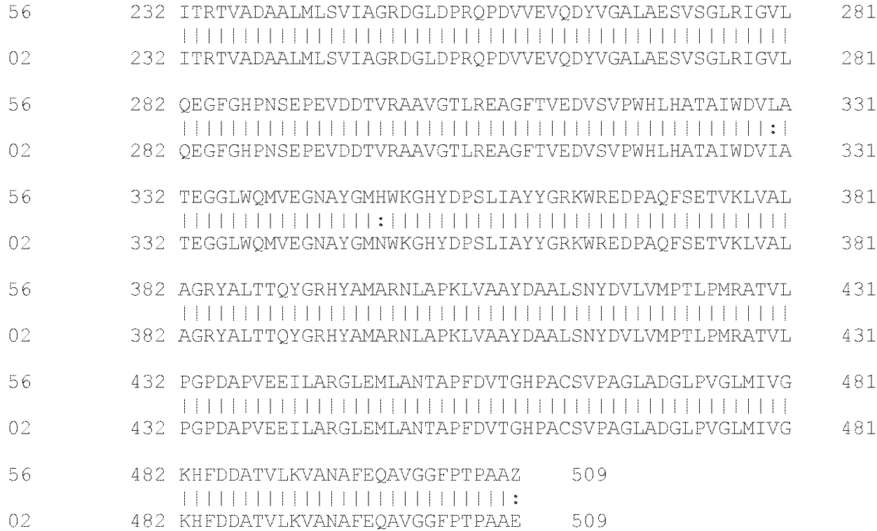

WO 2004/083423 A1 relates to thermally stable amidases isolated from thermophilic organisms, in particular amidases from thermophilic actinomycetes such as Pseudonocardia thermophilia. According to the sequence listing disclosed therein, the amino acid sequence of an amidase purported to be derived from Pseudonocardia thermophilia is disclosed as SEQ ID NO. 3 in WO 2004/083423 A1 (corresponding to SEQ ID NO. 56 of this application). The genome of Pseudonocardia thermophilia has since then been completely sequenced and deposited under GenBank number FRAP01000003.1 (https://www.ncbi.nlm.nih.gov/nuccore/ FRAP01000003). The genome has a gene locus of an amidase in the range 50704-52245; the protein is deposited under GenBank SHK14489.1. A sequence alignment of the deposited amidase compared to the wild-type amidase of SEQ ID NO.2 (not according to the invention) shows an identity of 100% over the entire length of the protein. However, the amino acid sequence according to WO 2004/083423 A1 appears to be incorrect, especially in the sequence section of the first approximately 100 N-terminal amino acid residues. These sequence inaccuracies may have arisen primarily due to the sequencing methods available at the time, so that SEQ ID NO. 3 of WO 2004/083423 A1 is erroneous from a present-day perspective. Thus, the correct amino acid sequence of the wild-type amidase from Pseudonocardia thermophilia is rather the present (non-inventive) SEQ ID NO. 2. A sequence alignment of SEQ ID NO. 3 according to WO 2004/083423 A1 in comparison to the wild-type amidase from Pseudonocardia thermophilia according to the present SEQ ID NO. 2 results in only 447 of 528 identical positions (Identity: 84.7%) as well as 458 of 528 similar positions (Similarity: 86.7%); the alignment is shown in FIG. 1.

The final report of the Technical University of Hamburg on the project, which is apparently related to the application WO 2004/083423, from December 2005 entitled “Use of amidases from extremophilic microorganisms for the enantioselective synthesis of amino and carboxylic acids AZ 13107” (https://www.dbu.de/projekt_13107/01_db_2409.html) contains supplementary information. According to the project description, the inventors of WO 2004/083423 A1 collaborated in this project. Accordingly, parts of the protein sequence of amidases isolated from Pseudonocardia thermophilia were sequenced and used to amplify and sequence the putative amidase gene in the organism by PCR. In experiments to produce the recombinant amidase by expression in E. coli host systems, the gene sequence amplified by PCR was cloned into the arabinose-induced expression vector pBad-Thio-TOPO. Expression of the protein with a size of 54 kDa could be detected by SDS-PAGE, but no activity of the recombinant amidase from Pseudonocardia thermophilia could be detected in enzymatic assays with substrates converted by the native protein. Thus, the disclosure of WO 2004/083423 A1 is not enabled for a person skilled in the art.

EP 0 272 024 A2 concerns a process for the decomposition of acrylamide using an amidase.

M. Cha, Eur Food Res Technol (2013) 236:567-571 concerns enzymatic control of acrylamide content in coffee using enzymes from Ralstonia eutropha and Geobacillus thermoglucasidasius. The publication does not name a specific enzyme sequence; currently known enzymes from Ralstonia eutropha have a maximum identity of about 36.4% and currently known enzymes from Geobacillus thermoglucasidasius have a maximum identity of about 53.3%, in each case compared to the enzyme from Pseudonoracdia thermophilia.

S. Raghavan et al, Microb Cell Fact (2019) 18:139 concerns the development and application of a transcriptional sensor to detect heterologous production of acrylic acid in E. coli. The paper names the amidase RAPc8 from Geobacillus pallidus, which has an identity of about 14% compared to the enzyme from Pseudonoracdia thermophilia and is unrelated to the latter.

T.K. Cheong et al, Enzyme and Microbial Technology 26 (2000) 152-158 concems the cloning of an amidase from Bacillus stearothermophilus BR388 in E. coli. The amidase from Bacillus stearothermophilus BR388 has an identity of about 14% compared to the enzyme from Pseudonoracdia thermophilia and is unrelated to the latter.

A. Karmali et al, Molecular Biotechnology, Vol. 17 2001 211-212 concerns the exchanges of Thr-103-Ile and Trp-138-Gly in amidase from Pseudomonas aeruginosa. The amidase from Pseudomonas aeruginosa has an identity of about 15% compared to the enzyme from Pseudonoracdia thermophilia and is unrelated to the latter.

N.J. Silman et al, J Gen Microbiol (1991) 137 169-178 concerns the undirected evolution of amidase-expessing Methylophilus methylotrophus by growth selection and chemical mutagenesis. This amidase has an identity of about 14% compared to the enzyme from Pseudonoracdia thermophilia and is unrelated to the latter.

M.S. Nawaz et al, J Bacteriol 1996 2397-2401 concems the physical, biochemical and immunological characterization of a thermostable amidase from Klebsiella pneumoniae. The publication does not name a specific enzyme sequence; currently known enzymes from Klebsiella pneumoniae have a maximum identity of about 51.2% compared to the enzyme from Pseudonoracdia thermophilia.

In all processes that use enzymes, the use of enzymes is associated with a precise setting of the process variables. For all enzymes, there is a temperature and pH range (including the respective optimum) at which the catalysis of the respective reaction takes place or can take place. Use outside these ranges usually results in the enzyme not being able to develop its catalytic function, or not to the desired extent. This is often the case, for example, at temperatures above 42° C. with non-thermophilic enzymes as well as at substantially acidic or clearly basic pH values. One difficulty in enzyme processes therefore always lies in the correct selection or modification of the enzymes so that they suit the process conditions.

The primary object of the present invention was to provide suitable enzymes and methods for the treatment of preparations containing acrylamide, which are capable of reducing the acrylamide content in preparations, preferably by at least 80 wt.% compared with the preparation before treatment, the enzymes preferably retaining their enzyme activity and/or exhibiting high stability even at high temperatures, such as those prevailing after steps of scalding, frying, roasting of food and luxury foods, and/or at a pH value between pH 4 and pH 7. Further objects underlying the present invention result from the following explanations and the appended claims.

The problem of the present invention is primarily solved by providing enzymes (as described herein and in particular in the claims), preferably amidases, which are indeed capable of significantly reducing the amount of acrylamide in a preparation, and this also at temperatures and pH values which are unfavorable for many amidases.

Furthermore, according to a preferred embodiment, the present invention relates to such enzymes (as described herein and in particular in the claims), preferably amidases, which are catalytically active up to a temperature of 50° C. or more as well as (also) in a pH range from pH 4 to pH 7.

In addition, a suitable method for degrading acrylamide in a preparation using an enzyme according to the invention (as described herein and in particular in the claims) is provided, as well as a method for preparing a preparation having a reduced acrylamide content.

Further provided are preparations having a reduced acrylamide content obtained by a process according to the invention.

Details of the present invention, preferred and alternative embodiments and aspects will be apparent from the following explanations, the appended sequences and, in particular, the appended claims.

FIG. 1 shows an alignment of SEQ ID NO. 65 (lines “65”) and SEQ ID NO. 2 (lines “02”): Identity: 447/528 (84.7%), Similarity: 458/528 (86.7%), Gaps: 41/528 (7.8%), Sequence Coverage: 507/513 (98.8%).

SEQ ID NO. 1 describes the amino acid consensus sequence of enzymes according to the invention.

SEQ ID NO. 2 describes the amino acid sequence (not according to the invention) of wild-type amidase from Pseudonocardia thermophila.

SEQ ID NO. 3 describes an amino acid sequence according to the invention, which contains a mutation at position 68 compared to SEQ ID NO. 2 (D68N).

SEQ ID NO. 4 describes an amino acid sequence according to the invention, which contains a mutation at position 74 compared to SEQ ID NO. 2 (A74Y).

SEQ ID NO. 5 describes an amino acid sequence according to the invention, which contains a mutation at position 445 compared to SEQ ID NO. 2 (G445A).

SEQ ID NO. 6 describes an amino acid sequence according to the invention, which contains a mutation at position 33 compared to SEQ ID NO. 2 (S33F).

SEQ ID NO. 7 describes an amino acid sequence according to the invention, which contains a mutation at position 33 compared to SEQ ID NO. 2 (S33R).

SEQ ID NO. 8 describes an amino acid sequence according to the invention, which contains a mutation at position 445 compared to SEQ ID NO. 2 (G445S).

SEQ ID NO. 9 describes an amino acid sequence according to the invention containing five mutations at positions 33, 74, 225, 445 and 453 compared to SEQ ID NO. 2 (S33R, A74Y, S225T, G445S, A453C).

SEQ ID NO. 10 describes an amino acid sequence according to the invention containing six mutations at positions 33, 68, 74, 225, 445 and 453 compared to SEQ ID NO. 2 (S33R, D68N, A74Y, S225T, G445S, A453C).

SEQ ID NO. 11 describes an amino acid sequence according to the invention containing five mutations at positions 33, 68, 74, 225 and 445 compared to SEQ ID NO. 2 (S33R, D68N, A74Y, S225T, G445A).

SEQ ID NO. 12 describes an amino acid sequence according to the invention containing four mutations at positions 68, 74, 445 and 453 compared to SEQ ID NO. 2 (D68N, A74Y, G445S, A453C).

SEQ ID NO. 13 describes an amino acid sequence according to the invention containing four mutations at positions 33, 68, 74 and 225 compared to SEQ ID NO. 2 (S33H, D68N, A74Y, S225T).

SEQ ID NO. 14 describes an amino acid sequence according to the invention containing twelve mutations at positions 33, 41, 68, 74, 94, 201, 225, 424, 445, 448, 453 and 507 compared to SEQ ID NO. 2 (S33R, W41Y, D68N, A74Y, V94I, Y201F, S225T, L424V, G445A, M448H, A453D, A507P).

SEQ ID NO. 15 describes an amino acid sequence according to the invention containing eleven mutations at positions 33, 68, 74, 94, 201, 225, 424, 445, 448, 453 and 507 compared to SEQ ID NO. 2 (S33R, D68N, A74Y, V94I, Y201F, S225T, L424V, G445A, M448H, A453D, A507P).

SEQ ID NO. 16 describes an amino acid sequence according to the invention containing eleven mutations at positions 33, 41, 68, 74, 94, 201, 225, 424, 445, 448 and 453 compared to SEQ ID NO. 2 (S33Y, W41Y, D68N, A74Y, V94I, Y201F, S225T, L424V, G445A, M448H, A453D).

SEQ ID NO. 17 describes an amino acid sequence according to the invention containing nine mutations at positions 33, 41, 68, 74, 201, 225, 424, 445, and 448 compared to SEQ ID NO. 2 (S33R, W41Y, D68N, A74Y, Y201F, S225T, L424V, G445A, M448H).

SEQ ID NO. 18 describes an amino acid sequence according to the invention containing twelve mutations at positions 33, 41, 68, 74, 94, 201, 225, 424, 445, 448, 453 and 507 compared to SEQ ID NO. 2 (S33R, W41Y, D68N, A74Y, V94I, Y201F, S225T, L424V, G445A, M448H, A453C, A507P).

SEQ ID NO. 19 describes an amino acid sequence according to the invention containing eleven mutations at positions 33, 41, 68, 74, 94, 201, 225, 424, 445, 448 and 453 compared to SEQ ID NO. 2 (S33Y, W41Y, D68N, A74Y, V94I, Y201F, S225T, L424V, G445A, M448H, A453N).

SEQ ID NO. 20 describes an amino acid sequence according to the invention containing eleven mutations at positions 33, 41, 68, 74, 94, 201, 225, 424, 445, 448 and 453 compared to SEQ ID NO. 2 (S33Y, W41Y, D68N, A74Y, V94I, Y201F, S225T, L424V, G445A, M448H, A453Q).

SEQ ID NO. 21 describes an amino acid sequence according to the invention containing eleven mutations at positions 33, 41, 68, 74, 94, 201, 225, 424, 445, 448 and 453 compared to SEQ ID NO. 2 (S33Y, W41Y, D68N, A74Y, V94I, Y201F, S225T, L424V, G445A, M448H, A453E).

SEQ ID NO. 22 describes an amino acid sequence according to the invention containing eleven mutations at positions 33, 41, 68, 74, 94, 201, 225, 424, 445, 448 and 453 compared to SEQ ID NO. 2 (S33Y, W41Y, D68N, A74Y, V94I, Y201F, S225T, L424V, G445A, M448H, A453K).

SEQ ID NO. 23 describes an amino acid sequence according to the invention containing six mutations at positions 33, 68, 74, 225, 445 and 454 compared to SEQ ID NO. 2 (S33R, D68N, A74Y, S225T, G445A, P454N).

SEQ ID NO. 24 describes an amino acid sequence according to the invention containing six mutations at positions 33, 68, 74, 225, 445 and 457 compared to SEQ ID NO. 2 (S33R, D68N, A74Y, S225T, G445A, V457G).

SEQ ID NO. 25 describes an amino acid sequence according to the invention containing six mutations at positions 33, 68, 74, 225, 424 and 445 compared to SEQ ID NO. 2 (S33R, D68N, A74Y, S225T, L424V, G445A).

SEQ ID NO. 26 describes an amino acid sequence according to the invention containing six mutations at positions 33, 68, 74, 225, 445 and 453 compared to SEQ ID NO. 2 (S33R, D68N, A74Y, S225T, G445A, A453D).

SEQ ID NO. 27 describes an amino acid sequence according to the invention containing five mutations at positions 33, 68, 74, 225, and 445 compared to SEQ ID NO. 2 (S33Y, D68N, A74Y, S225T, G445A).

SEQ ID NO. 28 describes an amino acid sequence according to the invention containing five mutations at positions 33, 68, 74, 175 and 445 compared to SEQ ID NO. 2 (S33R, D68N, A74Y, G175A, S225T, G445A).

SEQ ID NO. 29 describes an amino acid sequence according to the invention containing six mutations at positions 33, 68, 74, 225, 445 and 507 compared to SEQ ID NO. 2 (S33R, D68N, A74Y, S225T, G445A, A507P).

SEQ ID NO. 30 describes an amino acid sequence according to the invention containing six mutations at positions 33, 68, 74, 225, 445 and 453 compared to SEQ ID NO. 2 (S33R, D68N, A74Y, S225T, G445A, A453S).

SEQ ID NO. 31 describes an amino acid sequence according to the invention containing six mutations at positions 33, 68, 74, 94, 225 and 445 compared to SEQ ID NO. 2 (S33R, D68N, A74Y, V94I, S225T, G445A).

SEQ ID NO. 32 describes an amino acid sequence according to the invention containing six mutations at positions 33, 68, 74, 225, 317 and 445 compared to SEQ ID NO. 2 (S33R, D68N, A74Y, S225T, V317I, G445A).

SEQ ID NO. 33 describes an amino acid sequence according to the invention containing six mutations at positions 33, 68, 74, 201, 225 and 445 compared to SEQ ID NO. 2 (S33R, D68N, A74Y, Y201F, S225T, G445A).

SEQ ID NO. 34 describes an amino acid sequence according to the invention containing five mutations at positions 33, 68, 74, 445 and 448 compared to SEQ ID NO. 2 (S33R, D68N, A74Y, S225T, G445A, M448H).

SEQ ID NO. 35 describes an amino acid sequence according to the invention containing six mutations at positions 33, 68, 74, 225, 445 and 453 compared to SEQ ID NO. 2 (S33R, D68N, A74Y, S225T, G445A, A453R).

SEQ ID NO. 36 describes an amino acid sequence according to the invention containing six mutations at positions 33, 68, 74, 221, 225 and 445 compared to SEQ ID NO. 2 (S33R, D68N, A74Y, P221G, S225T, G445A).

SEQ ID NO. 37 describes an amino acid sequence according to the invention containing six mutations at positions 33, 68, 74, 217, 225 and 445 compared to SEQ ID NO. 2 (S33R, D68N, A74Y, T217R, S225T, G445A).

SEQ ID NO. 38 describes an amino acid sequence according to the invention containing six mutations at positions 33, 68, 74, 225, 328 and 445 compared to SEQ ID NO. 2 (S33R, D68N, A74Y, S225T, D328R, G445A).

SEQ ID NO. 39 describes an amino acid sequence according to the invention containing five mutations at positions 33, 68, 74, 225 and 445 compared to SEQ ID NO. 2 (S33R, D68N, A74Y, S225T, G445S).

SEQ ID NO. 40 describes an amino acid sequence according to the invention containing six mutations at positions 33, 68, 74, 225, 229 and 445 compared to SEQ ID NO. 2 (S33R, D68N, A74Y, S225T, L229C, G445A).

SEQ ID NO. 41 describes an amino acid sequence according to the invention containing six mutations at positions 33, 41, 68, 74, 225 and 445 compared to SEQ ID NO. 2 (S33R, W41Y, D68N, A74Y, S225T, G445A).

SEQ ID NO. 42 describes an amino acid sequence according to the invention, which contains a mutation at position 225 compared to SEQ ID NO. 2 (S225T).

SEQ ID NO. 43 describes an amino acid sequence according to the invention, which contains a mutation at position 33 compared to SEQ ID NO. 2 (S33Y).

SEQ ID NO. 44 describes an amino acid sequence according to the invention, which contains a mutation at position 317 compared to SEQ ID NO. 2 (V317I).

SEQ ID NO. 45 describes an amino acid sequence according to the invention, which contains a mutation at position 453 compared to SEQ ID NO. 2 (A453E).

SEQ ID NO. 46 describes an amino acid sequence according to the invention, which contains a mutation at position 33 compared to SEQ ID NO. 2 (S33H).

SEQ ID NO. 47 describes an amino acid sequence according to the invention, which contains a mutation at position 33 compared to SEQ ID NO. 2 (S33H).

SEQ ID NO. 48 describes an amino acid sequence according to the invention, which contains a mutation at position 453 compared to SEQ ID NO. 2 (A453Q).

SEQ ID NO. 49 describes an amino acid sequence according to the invention, which contains a mutation at position 424 compared to SEQ ID NO. 2 (L424V).

SEQ ID NO. 50 describes an amino acid sequence according to the invention, which contains a mutation at position 453 compared to SEQ ID NO. 2 (A453C).

SEQ ID NO. 51 describes an amino acid sequence according to the invention, which contains a mutation at position 453 compared to SEQ ID NO. 2 (A453K).

SEQ ID NO. 52 describes an amino acid sequence according to the invention, which contains a mutation at position 453 compared to SEQ ID NO. 2 (A453S).

SEQ ID NO. 53 describes an amino acid sequence according to the invention, which contains a mutation at position 454 compared to SEQ ID NO. 2 (A454N).

SEQ ID NO. 54 describes an amino acid sequence according to the invention, which contains a mutation at position 507 compared to SEQ ID NO. 2 (A507P).

SEQ ID NO. 55 describes an amino acid sequence according to the invention, which contains a mutation at position 453 compared to SEQ ID NO. 2 (A453N).

SEQ ID NO. 56 corresponds to the protein sequence of Pseudonocardia thermophila as disclosed as “SEQ ID NO. 3” in WO 2004/083423.

The amino acid exchanges of SEQ ID NO. 3 to 55 according to the invention compared with SEQ ID NO.2 (wild-type amidase from Pseudonocardia thermophila) are summarized in the following table:

| SEQ ID | S33 | W41 | D68 | A74 | V94 | G175 | Y201 | T217 | P221 | S225 | L229 | V317 | D328 | L424 | G445 | M448 | A453 | P454 | C457 | A507 |
|---|---|---|---|---|---|---|---|---|---|---|---|---|---|---|---|---|---|---|---|---|
| 3 | | | N | | | | | | | | | | | | | | | | | |
| 4 | | | | Y | | | | | | | | | | | | | | | | |
| 5 | | | | | | | | | | | | | | | A | | | | | |
| 6 | F | | | | | | | | | | | | | | | | | | | |
| 7 | R | | | | | | | | | | | | | | | | | | | |
| 8 | | | | | | | | | | | | | | | S | | | | | |
| 9 | R | | | Y | | | | | | T | | | | | S | | C | | | |
| 1 | R | | N | Y | | | | | | T | | | | | S | | C | | | |
| 1 | R | | N | Y | | | | | | T | | | | | A | | | | | |
| 1 | | | N | Y | | | | | | | | | | | S | | C | | | |
| 1 | H | | N | Y | | | | | | T | | | | | | | | | | |
| 1 | R | Y | N | Y | I | | F | | | T | | | | V | A | H | D | | | P |
| 1 | R | | N | Y | I | | F | | | T | | | | V | A | H | D | | | P |
| 1 | Y | Y | N | Y | I | | F | | | T | | | | V | A | H | D | | | |
| 1 | R | Y | N | Y | | | F | | | T | | | | V | A | H | | | | |
| 1 | R | Y | N | Y | I | | F | | | T | | | | V | A | H | C | | | P |
| 1 | Y | Y | N | Y | I | | F | | | T | | | | V | A | H | N | | | |
| 2 | Y | Y | N | Y | I | | F | | | T | | | | V | A | H | Q | | | |
| 2 | Y | Y | N | Y | I | | F | | | T | | | | V | A | H | E | | | |
| 2 | Y | Y | N | Y | I | | F | | | T | | | | V | A | H | K | | | |
| 2 | R | | N | Y | | | | | | T | | | | | A | | | N | | |
| 2 | R | | N | Y | | | | | | T | | | | | A | | | | G | |
| 2 | R | | N | Y | | | | | | T | | | | V | A | | | | | |
| 2 | R | | N | Y | | | | | | T | | | | | A | | D | | | |
| 2 | Y | | N | Y | | | | | | T | | | | | A | | | | | |
| 2 | R | | N | Y | | A | | | | T | | | | | A | | | | | |
| 2 | R | | N | Y | | | | | | T | | | | | A | | | | | P |
| 3 | R | | N | Y | | | | | | T | | | | | A | | S | | | |
| 3 | R | | N | Y | I | | | | | T | | | | | A | | | | | |
| 3 | R | | N | Y | | | | | | T | | I | | | A | | | | | |
| 3 | R | | N | Y | | | F | | | T | | | | | A | | | | | |
| 3 | R | | N | Y | | | | | | T | | | | | A | H | | | | |
| 3 | R | | N | Y | | | | | | T | | | | | A | | R | | | |
| 3 | R | | N | Y | | | | | G | T | | | | | A | | | | | |
| 3 | R | | N | Y | | | | R | | T | | | | | A | | | | | |
| 3 | R | | N | Y | | | | | | T | | | R | | A | | | | | |
| 3 | R | | N | Y | | | | | | T | | | | | S | | | | | |
| 4 | R | | N | Y | | | | | | T | C | | | | A | | | | | |
| 4 | R | Y | N | Y | | | | | | T | | | | | A | | | | | |
| 4 | | | | | | | | | | T | | | | | | | | | | |
| 4 | Y | | | | | | | | | | | | | | | | | | | |
| 4 | | | | | | | | | | | | I | | | | | | | | |
| 4 | | | | | | | | | | | | | | | | | E | | | |
| 4 | H | | | | | | | | | | | | | | | | | | | |
| 4 | | | | | | A | | | | | | | | | | | | | | |
| 4 | | | | | | | | | | | | | | | | | Q | | | |
| 4 | | | | | | | | | | | | | | V | | | | | | |
| 5 | | | | | | | | | | | | | | | | | C | | | |
| 5 | | | | | | | | | | | | | | | | | K | | | |
| 5 | | | | | | | | | | | | | | | | | S | | | |
| 5 | | | | | | | | | | | | | | | | | | N | | |
| 5 | | | | | | | | | | | | | | | | | | | | P |
| 5 | | | | | | | | | | | | | | | | | N | | | |

In a first aspect of the present invention, there is provided an enzyme for reducing the amount of acrylamide in a preparation comprising or consisting of an amino acid consensus sequence according to SEQ ID NO. 1, wherein the amino acid consensus sequence is not a sequence according to SEQ ID NO. 2, and wherein the enzyme comprises or consists of an amino acid sequence having a sequence identity of at least 95%, 96%, 97%, 98% or 99% to a sequence selected from the group consisting of the sequences set forth in SEQ ID NO. 3 to SEQ ID NO. 55.

As mentioned above, SEQ ID NO. 1 describes the amino acid consensus sequence of enzymes according to the invention. A consensus sequence describes the amino acid sequence possessed by all enzymes according to the invention. The variable positions are indicated by Xaa and represent positions in which the enzymes according to the invention may differ from each other.

Enzymes are biological catalysts that catalyze a specific chemical reaction. In the present case, the degradation of acrylamide by an amidase, the amide bond of the acrylamide is hydrolytically cleaved to form acrylic acid and ammonia. The concentrations of acrylic acid and ammonia or, if applicable, substances resulting from them (in the case of further reaction of acrylic acid or ammonia) are so low in coffee preparation that they have no negative effects on the end user. For example, ammonia reacts immediately to form harmless ammonium, which has no effect on the end product.

Whenever the present disclosure refers to sequence identities of amino acid sequences in terms of percentages, such references refer to values as can be calculated using EMBOSS Water Pairwise Sequence Alignments (Nucleotide) (http://www.ebi.ac.uk/Tools/psa/emboss_water/nucleotide.html) for nucleic acid sequences or EMBOSS Water Pairwise Sequence Alignments (Protein) (http://www.ebi.ac.uk/Tools/psa/emboss_water/) for amino acid sequences. In the case of the local sequence alignment tools provided by the European Molecular Biology Laboratory (EMBL) European Bioinformatics Institute (EBI), a modified Smith-Waterman algorithm is used (see http://www.ebi.ac.uk/Tools/psa/ and Smith, T.F. & Waterman, M.S. “identification of common molecular subsequences” Journal of Molecular Biology, 1981 147 (1):195-197). Furthermore, here, when performing the respective pairwise alignment of two sequences using the modified Smith-Waterman algorithm, reference is made to the default parameters currently given by EMBL-EBI. These are (i) for amino acid sequences: Matrix = BLOSUM62, Gap open penalty = 10 and Gap extend penalty = 0.5 and (ii) for nucleic acid sequences: Matrix = DNAfull, Gap open penalty = 10 and Gap extend penalty = 0.5. In addition to the default parameters, when aligning a sequence of interest (“Query Sequence”, for EMBOSS the first sequence) with a reference sequence (“Subject Sequence”, for EMBOSS the second sequence), the Subject Sequence must be represented at least 93% over the length of the single alignment (“Sequence Coverage” at least 93%); alignments with lower Sequence Coverage of the Subject Sequence are excluded for determining sequence identity for the purposes of this application. However, the Query Sequence may be longer than the length of the Alignment, and the sequences represented in the Alignment of the Query Sequence may be above or below 93%. For example, 507 amino acids of the total 513 amino acids of the subject sequence occur in the alignment of SEQ ID NO. 56 as the query sequence with SEQ ID NO. 2 as the subject sequence (FIG. 1); thus, the sequence coverage is 507/513 (98.8%).

The term “sequence identity” can therefore be used interchangeably with “sequence homology” in the context of the present invention. The latter always refers to the total length of an enzyme according to the invention compared to the total length of an enzyme to which the sequence identity or sequence homology is determined.

In the context of the present invention, a preparation means any raw, semi-finished or finished food, luxury food or cosmetics, which includes, by way of example, fried or deep-fried potato products, roasted cereal or products containing roasted cereal, corn products, coffee products, e.g., solid or liquid coffee extract and green coffee, chicory extract, cereal coffee products, coffee substitute products, snacks, wheat products, cosmetics, bakery products or pastries, e.g., cookies, biscuits, rusks, cereal bars, scones, ice cream cones, waffles, crumpets, gingerbread, crispbread and bread substitutes, pasta, rice, fish products, meat products, cereals, beer, nuts, complementary foods for children and infants, hair styling products, personal care products, hair care products, and facial care products.

In the context of the present invention, an enzyme according to the invention does not comprise or consist of a sequence according to SEQ ID NO. 2. SEQ ID NO. 2 describes the amino acid sequence of the wild-type enzyme from Pseudonocardia thermophila from which the enzymes according to the invention are derived.

A homology model of the amino acid sequence according to SEQ ID NO. 2, i.e. of the wild-type enzyme from Pseudonocardia thermophila was built. YASARA structure version 20.10.4.L.64 software was used for this purpose with the included macro hm_build.mcr. The default settings were kept. The crystal structures 3A1K, 3A1I, 3IP4, and 2GI3 (https://www.rcsb.org/) were used as templates for the final homology model, which is largely based on the homodimeric structure 3A1K underlying the amidase from Rhodococcus sp. N-771, present in its catalytically active form as a homo-dimer (https://www.rcsb.org/structure/3a1k). Without wishing to be bound by any scientific theory, it is assumed that the amino acid sequence according to SEQ ID NO. 2, i.e. the wild-type enzyme from Pseudonocardia thermophila, has a folded structure of the type PDB 3A1K. To the alpha-C atom of the catalytic serine (194), the following amino acid residues have the following spatial distance: A74 (17.1 Å), D68 (21.0 Å), A507 (23.2 Å), G445 (10.6 Å), L424 (11.6 Å), and S33 (21.8 Å). Among themselves, the following residues have the following spatial distance: G445 to L424 (13.1 Å), G445 to S33 (12.0 Å), D68 to A74 (8.3 Å), D68 to A507 (16.0 Å). Therefore, within a space sphere around G445 with a radius of about 13 Å lie the active center as well as L424 and S33. Residues D68 and A74 lie in a loop structure within a space sphere with a radius of 4.2 Å (from the geometric center of both amino acids) or in a space sphere with a radius of 6.5 Å starting from C-alpha atom of A74. All named distances refer to the C-alpha atoms of the amino acids.

Furthermore, according to a preferred embodiment, in the context of the present invention, it is generally not necessary to use a wild-type enzyme from Pseudonocardia thermophila. That is, according to one preferred embodiment, a sequence according to SEQ ID NO. 1 is not only not a sequence according to SEQ ID NO. 2, but is generally not a sequence of a wild-type amidase from Pseudonocardia thermophila.

Other (thermostable) amidases from Pseudonocardia thermophila are described, for example, in WO 2004/083423 (EP 1608746 B1), but these are not enzymes according to the present invention and can also not be regarded as wildtype enzyme according to SEQ ID NO. 2. Moreover, WO 2004/083423 (EP 1608746) does not demonstrate that the enzymes described therein can advantageously be used in the food or luxury food sector in the sense of the present invention, in particular not under such temperature and/or pH conditions as described herein.

Enzymes according to the invention can be obtained starting from the wild-type enzyme by performing one or more steps (preferably of directed, or alternatively of undirected, or of directed and undirected) mutation. Directed mutation is the targeted alteration of one or more DNA bases of the enzyme gene, resulting in one or more targeted effects on the amino acid sequence. In contrast, undirected mutation is random mutagenesis in a portion of the entire DNA sequence that is not precisely selected. Following the undirected mutagenesis, the resulting protein is examined to determine if it has the desired properties. The enzyme to be modified may be a wild-type enzyme. This was the case in the considerations and investigations that led to the present invention. In the context of the present invention, a wild-type enzyme is understood to be a naturally occurring, technically unmodified enzyme which has been isolated from nature either as a functional enzyme or the sequence thereof, and wherein the sequence and therefore also the functional enzyme have not been modified by human hand. In one embodiment, the enzyme may be the product of iterative undirected mutagenesis. In another embodiment, the enzyme may be the product of iterative directed mutagenesis.

In the course of the studies underlying the invention, more than 2000 different mutants were generated starting from a wild-type enzyme, all having different mutations at different sites of the enzyme gene. These mutants were then tested for their activity as well as stability at different pH values and temperatures. From this, in a further mutagenesis step, the enzyme could be modified in such a way that increased activity and stability resulted compared to the wild-type enzyme. The amino acid residues relevant for catalysis were not mutated, in particular the catalytic triad of the enzyme consisting of a lysine at position 95, a serine at position 170 and a serine at position 194 were not mutated.

In the context of the present invention, it generally applies to an enzyme according to the invention that in the amino acid sequence thereof one, several or all, preferably all, of these three positions are not mutated, respectively at position 95 of the sequences according to the invention described herein is preferably a lysine, and/or at position 170 of the sequences according to the invention described herein is preferably a serine, and/or at position 194 of the sequences according to the invention described herein is preferably a serine, preferably at position 95 is a lysine, at position 170 is a serine and at position 194 is a serine.

In the context of the present invention, activity refers to the ability of the enzyme to catalyze the hydrolytic cleavage of the amide bond per unit time, whereas stability defines the residual activity of an enzyme under various environmental conditions, such as pH and temperature, after a certain time. Suitable mutagenesis methods as well as the necessary conditions and reagents are sufficiently known to the skilled person. Mutations take place at the gene level, for example, through the replacement (substitution), removal (deletion), or addition of bases. These mutations have different effects on the amino acid sequence of the resulting protein. In the case of substitution, so-called “nonsense” mutations can occur, causing protein biosynthesis to stop early and the resulting protein to remain dysfunctional. In the so-called “missense” mutation, only the encoded amino acid changes; these mutations result in a functional change in the resulting protein and, in the best case, may cause improved stability or activity of the resulting protein. In general nomenclature, amino acid substitution mutations are designated based on their position and the amino acid substituted, for example, as A143G. This notation means that at position 143 of the N- to C-terminal amino acid sequence, the amino acid alanine has been exchanged for guanine. It is very particularly preferred in the context of the present invention if substitution mutations are generated.

In a preferred embodiment of the present invention, the amino acid sequence (i.e., the amino acid sequence comprised by an enzyme according to the invention or constituting an enzyme according to the invention, having a sequence identity of at least 90%, 91%, 92%, 93%, 94%, 95%, 96%, 97%, 98% or 99% to a sequence according to SEQ ID NO. 1 or to a sequence according to SEQ ID NO. 1, wherein the amino acid sequence is not a sequence according to SEQ ID NO. 2) has, at least at one, more or all positions selected from the group consisting of positions 33, 41, 68, 74, 94, 175, 201, 217, 221, 225, 229, 317, 328, 424, 445, 448, 453, 454, 457 and 507, an amino acid which does not correspond to the amino acid of the corresponding position(s) of the amino acid sequence according to SEQ ID NO. 2.

In another embodiment of the present invention, the amino acid sequence has, at least at one, more or all of the positions selected from the group consisting of positions 33, 41, 68, 74, 94, 175, 201, 225, 317, 424, 445, 448, 453, 454 and 507, an amino acid other than the amino acid of the corresponding position(s) of the amino acid sequence according to SEQ ID NO. 2.

In another embodiment of the present invention, the amino acid sequence has, at least at one, more, or all of the positions selected from the group consisting of positions 33, 68, 74, 175, 201, 225, 317, 424, 445, 448, 453, and 507, an amino acid other than the amino acid of the corresponding position(s) of the amino acid sequence according to SEQ ID NO. 2.

In another embodiment of the present invention, the amino acid sequence has, at least at one, more, or all of the positions selected from the group consisting of positions 33, 68, 74, 201, 225, 424, 445, 448, 453, and 507, an amino acid other than the amino acid of the corresponding position(s) of the amino acid sequence according to SEQ ID NO. 2.

In another embodiment of the present invention, the amino acid sequence has, at least at one, more or all of the positions selected from the group consisting of positions 33, 68, 74, 175, 225, 317, 424, 445, 453, 454 and 507, an amino acid other than the amino acid of the corresponding position(s) of the amino acid sequence according to SEQ ID NO. 2.

In another embodiment of the present invention, the amino acid sequence has, at least at one, more, or all of the positions selected from the group consisting of positions 33, 68, 225, 424, 445, 453, and 507, an amino acid that does not correspond to the amino acid of the corresponding position(s) of the amino acid sequence according to SEQ ID NO. 2.

In another embodiment of the present invention, the amino acid sequence has, at least at one, more, or all of the positions selected from the group consisting of positions 33, 424, and 445, an amino acid that does not correspond to the amino acid of the corresponding position(s) of the amino acid sequence according to SEQ ID NO. 2.

In another embodiment of the present invention, the amino acid sequence has, at least at one, more, or all of the positions selected from the group consisting of positions 68, and 74, an amino acid that does not correspond to the amino acid of the corresponding position(s) of the amino acid sequence according to SEQ ID NO. 2.

In another embodiment of the present invention, the amino acid sequence has, at position 33, an arginine or a tyrosine or a histidine or a phenylalanine, and/or at position 41 a tyrosine, and/or at position 68 an asparagine, and/or at position 74 a tyrosine, and/or at position 94 an isoleucine, and/or at position 175 an alanine, and/or at position 201 a phenylalanine, and/or at position 217 an arginine, and/or at position 221 a glycine, and/or at position 225 a threonine, and/or at position 229 a cysteine, and/or at position 317 an isoleucine, and/or at position 328 an arginine, and/or at position 424 a valine, and/or at position 445 an arginine or a serine, and/or at position 448 a histidine, and/or at position 453, an aspartate or a cysteine or an asparagine or a glutamine or a glutamate or a lysine or an arginine or a serine, and/or at position 454 an asparagine, and/or at position 457 a glycine, and/or at position 507 a proline.

In another embodiment of the present invention, the amino acid sequence has, at position 33 an arginine or a tyrosine, and/or at position 68 an asparagine, and/or at position 74 a tyrosine, and/or at position 201 a phenylalanine, and/or at position 225 a threonine, and/or at position 424 a valine, and/or at position 445 an alanine, and/or at position 448 a histidine, and/or at position 453 an aspartate or a cysteine.

From the preferred amino acids at the positions described above, the following preferred embodiments or amino acid substitutions arise when starting from the wild-type sequence according to SEQ ID NO. 2:

In one embodiment of the present invention, the amino acid sequence has at least one amino acid substitution selected from the group consisting of S33R, S33Y, S33H, S33F, particularly preferably an amino acid substitution S33R, S33H or S33Y.

In another embodiment of the present invention, the amino acid sequence has at least one amino acid substitution W41Y.

In a preferred embodiment of the present invention, the amino acid sequence has at least one amino acid substitution D68N.

According to another preferred embodiment of the present invention, the amino acid sequence has at least one amino acid substitution A74Y.

In another embodiment of the present invention, the amino acid sequence has at least one amino acid substitution V94I.

In another embodiment of the present invention, the amino acid sequence has at least one amino acid substitution G175A.

In another embodiment of the present invention, the amino acid sequence has at least one amino acid substitution Y201F.

According to another embodiment of the present invention, the amino acid sequence has at least one amino acid substitution T217R.

In another embodiment of the present invention, the amino acid sequence has at least one amino acid substitution P221G.

In another preferred embodiment of the present invention, the amino acid sequence has at least one amino acid substitution S225T.

According to another embodiment of the present invention, the amino acid sequence has at least one amino acid substitution L229C.

In yet another embodiment of the present invention, the amino acid sequence has at least one amino acid substitution V317I.

In another embodiment of the present invention, the amino acid sequence has at least one amino acid substitution D328R.

According to another embodiment of the present invention, the amino acid sequence has at least one amino acid substitution L424V.

In another embodiment of the present invention, the amino acid sequence has at least one amino acid substitution selected from the group consisting of G445A and G445S, preferably an amino acid substitution G445A.

According to another embodiment of the present invention, the amino acid sequence has at least one amino acid substitution M448H.

In another embodiment of the present invention, the amino acid sequence has at least one amino acid substitution selected from the group consisting of A453D, A543C, A453N, A453Q, A453E, A453K, A453R, and A453S, particularly preferably an amino acid substitution A453D, A453Q, A453N or A453C.

According to another embodiment of the present invention, the amino acid sequence has at least one amino acid substitution P454N.

According to yet another embodiment of the present invention, the amino acid sequence has at least one amino acid substitution V457G.

In another embodiment of the present invention, the amino acid sequence has at least one amino acid substitution A507P.

From the present text, it naturally follows for the person skilled in the art that one or more of the amino acid substitutions described herein, in particular those described above, or the amino acids preferably present at the respective positions, can be combined with one another in any desired manner in order to obtain enzymes according to the invention, if necessary in combination with further substitutions not described herein or amino acids other than the amino acids according to SEQ ID NO. 1 (cf. in this respect the “sequence identity of at least 90%, 91%, 92%, 93%, 94%, 95%, 96%, 97%, 98% or 99% to a sequence according to SEQ ID NO. 1” described according to the invention). In the context of the present invention, those amino acid substitutions are preferred here which lie outside functional, in particular outside catalytic (cf. above), regions.

Another aspect of the invention relates to an enzyme which is not described with reference to the consensus sequence according to SEQ ID NO. 1, but independently thereof. This further aspect of the invention relates to an enzyme for reducing the amount of acrylamide in a preparation comprising an amino acid sequence having a sequence identity of at least 86%, 87%, 88%, 89%, 90%, 91%, 92%, 93%, 94%, 95%, 96%, 97%, 98% or 99% to SEQ ID NO. 2, wherein the amino acid sequence compared to SEQ ID NO. 2 has at least one amino acid substitution in a position located in one of the following sequence segments of SEQ ID NO. 2:
(a) Position L424 to position A507; preferably in position G445;
(b) position S33 to position A74; or
(c) Position G175 to position L229.

In a particularly preferred embodiment, the enzyme according to the invention comprises an amino acid sequence having a sequence identity of at least 90%, 91%, 92%, 93%, 94%, 95%, 96%, 97%, 98% or 99% to SEQ ID NO. 2, wherein the amino acid sequence has at least the following amino acid substitutions compared to SEQ ID NO. 2:
S33G, S33A, S33V, S33L, S33I, S33M, S33P, S33F, S33W, S33Y, S33S, S33T, S33C, S33N, S33Q, S33D, S33E, S33R, S33K or S33H, preferably S33R, S33Y, S33H or S33F
G445A, G445V, G445L, G445I, G445M, G445P, G445F, G445W, G445Y, G445S, G445T, G445C, G445N, or G445Q; preferably G445A, or G445S; and
L424G, L424A, L424V, L424I, L424M, L424P, L424F or L424W; preferably L424V.

For this further aspect of the invention, the provisions on the determination of sequence identities of amino acid sequences in the form of percentages according to the above aspects and embodiments apply unchanged.

In preferred embodiments, compared to an enzyme having SEQ ID NO. 2, the enzyme according to the invention exhibits
(i) higher enzyme activity in the catalyzed release of ammonia from 25 mM acrylamide in 50 mM sodium acetate buffer pH 5.5 at 40° C.;
(ii) higher enzyme activity in the catalyzed release of ammonia from 25 mM acrylamide in 50 mM sodium acetate buffer pH 5 at 40° C.;
(iii) higher Tₘ 50 value in 50 mM sodium acetate buffer pH 5.5;
(iv) higher Tₘ 50 value in 50 mM sodium acetate buffer pH 5; and/or
(v) higher residual activity after incubation for 24 h at 50° C. in 50 mM sodium acetate buffer pH 5.0.

A higher (enzyme) activity means a higher activity of an enzyme (as defined above) in a sample compared to another enzyme in corresponding sample. Accordingly, a higher residual activity (of an enzyme) means the activity of an enzyme (as defined above) in a sample after a specified time in comparison to another enzyme in corresponding sample. The determination of the activity, residual activity and/or Tm value is preferably carried out as described in the experimental section.

Compared to SEQ ID NO. 2, the enzyme according to the invention has at least one amino acid exchange (substitution), preferably at least two amino acid exchanges, more preferably at least three amino acid exchanges, more preferably at least four amino acid exchanges, more preferably at least five amino acid exchanges, more preferably at least six amino acid exchanges, more preferably at least seven amino acid exchanges, more preferably at least eight amino acid exchanges, more preferably at least nine amino acid exchanges, even more preferably at least ten amino acid exchanges, and most preferably at least eleven amino acid exchanges.

Compared to SEQ ID NO. 2, the enyme according to the invention has at most 23 amino acid exchanges, preferably at most 22 amino acid exchanges, more preferably at most 21 amino acid exchanges, more preferably at most 20 amino acid exchanges, more preferably at most 19 amino acid exchanges, more preferably at most 18 amino acid exchanges, more preferably at most 17 amino acid exchanges, more preferably at most 16 amino acid exchanges, more preferably at most 15 amino acid exchanges, even more preferably at most 14 amino acid exchanges, and most preferably at most 13 amino acid exchanges.

In a preferred embodiment of this aspect of the invention and its preceding embodiments, the amino acid sequence of the enzyme of the invention additionally has at least one amino acid substitution selected from the group consisting of positions V94, V317, and D328 compared to SEQ ID NO. 2.

In preferred embodiments, the amino acid sequence of the enzyme of the invention has at least two amino acid substitutions compared to SEQ ID NO.2, of which
the first amino acid exchange is located in a position in sequence section (a) and the second amino acid exchange is also located in a position in sequence section (a);
the first amino acid exchange is located in a position in sequence section (a) and the second amino acid exchange is located in a position in sequence section (b);
the first amino acid exchange is located in a position in sequence section (a) and the second amino acid exchange is located in a position in sequence section (c);
the first amino acid exchange is located in a position in sequence section (b) and the second amino acid exchange is also located in a position in sequence section (b);
the first amino acid exchange is located in a position in sequence section (b) and the second amino acid exchange is located in a position in sequence section (c); or
the first amino acid exchange is located in a position in sequence section (c) and the second amino acid exchange is also located in a position in sequence section (c).

In preferred embodiments, the amino acid sequence of the enzyme of the invention has at least two amino acid substitutions compared to SEQ ID NO.2, of which
the first amino acid exchange is located in a position in sequence section (a) and the second amino acid exchange is located in a position in sequence section (b);
the first amino acid exchange is located in a position in sequence section (a) and the second amino acid exchange is located in a position in sequence section (c); or
the first amino acid exchange is located in a position in sequence section (b) and the second amino acid exchange is located in a position in sequence section (c).

In preferred embodiments, the amino acid sequence of the enzyme of the invention has at least three amino acid substitutions compared to SEQ ID NO. 2, of which
the first amino acid exchange is located in a position in sequence section (a), the second amino acid exchange is also located in a position in sequence section (a), and the third amino acid exchange is also located in a position in sequence section (a);
the first amino acid exchange is located in a position in sequence section (a), the second amino acid exchange is also located in a position in sequence section (a), and the third amino acid exchange is located in a position in sequence section (b);
the first amino acid exchange is located in a position in sequence section (a), the second amino acid exchange is also located in a position in sequence section (a), and the third amino acid exchange is located in a position in sequence section (c);
the first amino acid exchange is located in a position in sequence section (a), the second amino acid exchange is located in a position in sequence section (b), and the third amino acid exchange is also located in a position in sequence section (b);
the first amino acid exchange is located in a position in sequence section (a), the second amino acid exchange is located in a position in sequence section (b), and the third amino acid exchange is located in a position in sequence section (c);
the first amino acid exchange is located in a position in sequence section (a), the second amino acid exchange is located in a position in sequence section (c), and the third amino acid exchange is also located in a position in sequence section (c);
the first amino acid exchange is located in a position in sequence section (b), the second amino acid exchange is also located in a position in sequence section (b), and the third amino acid exchange is also located in a position in sequence section (b);
the first amino acid exchange is located in a position in sequence section (b), the second amino acid exchange is also located in a position in sequence section (b), and the third amino acid exchange is located in a position in sequence section (c); or
the first amino acid exchange is located in a position in sequence section (c), the second amino acid exchange is also located in a position in sequence section (c), and the third amino acid exchange is also located in a position in sequence section (c).

In preferred embodiments, the amino acid sequence of the enzyme of the invention has at least three amino acid substitutions compared to SEQ ID NO. 2, of which
the first amino acid exchange is located in a position in sequence section (a), the second amino acid exchange is located in a position in sequence section (b), and the third amino acid exchange is located in a position in sequence section (b); or
the first amino acid exchange is located in a position in sequence portion (a), the second amino acid exchange is located in a position in sequence portion (a), and the third amino acid exchange is located in a position in sequence portion (b).

In preferred embodiments, the amino acid sequence of the enzyme of the invention, as compared to SEQ ID NO. 2, has at least one amino acid substitution in a position located in one of the following sequence segments of SEQ ID NO. 2:
(a-1) Position G445 to V465;
(a-2) Position L424 to R444;
(a-3) Item P487 to A507; or
(a-4) Position P466 to A486.

In preferred embodiments, the amino acid sequence of the enzyme of the invention, as compared to SEQ ID NO. 2, has at least one amino acid substitution in a position located in one of the following sequence segments of SEQ ID NO. 2:
(b-1) position S33 to position P53; or
(b-2) Position T54 to position A74.

In preferred embodiments, the amino acid sequence of the enzyme of the invention, as compared to SEQ ID NO. 2, has at least one amino acid substitution in a position located in one of the following sequence segments of SEQ ID NO. 2:
(c-1) Position G212 to position L229;
(c-2) Position G193 to position H211; or
(c-3) Position G175 to position G192.

In preferred embodiments, the amino acid sequence of the enzyme of the invention has at least two amino acid substitutions compared to SEQ ID NO. 2, of which
the first amino acid exchange is located in a position in sequence section (a-1) and the second amino acid exchange is also located in a position in sequence section (a-1);
the first amino acid exchange is located in a position in sequence section (a-1) and the second amino acid exchange is located in a position in sequence section (a-2);
the first amino acid exchange is located in a position in sequence section (a-1) and the second amino acid exchange is located in a position in sequence section (a-3);
the first amino acid exchange is located in a position in sequence section (a-1) and the second amino acid exchange is located in a position in sequence section (b-1);
the first amino acid exchange is located in a position in sequence section (a-1) and the second amino acid exchange is located in a position in sequence section (b-2);
the first amino acid exchange is located in a position in sequence section (a-1) and the second amino acid exchange is located in a position in sequence section (c-1);
the first amino acid exchange is located in a position in sequence section (a-1) and the second amino acid exchange is located in a position in sequence section (c-2);
the first amino acid exchange is located in a position in sequence section (a-2) and the second amino acid exchange is located in a position in sequence section (a-3);
the first amino acid exchange is located in a position in sequence section (a-2) and the second amino acid exchange is located in a position in sequence section (b-1);
the first amino acid exchange is located in a position in sequence section (a-2) and the second amino acid exchange is located in a position in sequence section (b-2);
the first amino acid exchange is located in a position in sequence section (a-2) and the second amino acid exchange is located in a position in sequence section (c-1);
the first amino acid exchange is located in a position in sequence section (a-2) and the second amino acid exchange is located in a position in sequence section (c-2);
the first amino acid exchange is located in a position in sequence section (a-3) and the second amino acid exchange is located in a position in sequence section (b-1);
the first amino acid exchange is located in a position in sequence section (a-3) and the second amino acid exchange is located in a position in sequence section (b-2);
the first amino acid exchange is located in a position in sequence section (a-3) and the second amino acid exchange is located in a position in sequence section (c-1);
the first amino acid exchange is located in a position in sequence section (a-3) and the second amino acid exchange is located in a position in sequence section (c-2);
the first amino acid exchange is located in a position in sequence section (b-1) and the second amino acid exchange is also located in a position in sequence section (b-1);
the first amino acid exchange is located in a position in sequence section (b-1) and the second amino acid exchange is located in a position in sequence section (b-2);
the first amino acid exchange is located in a position in sequence section (b-1) and the second amino acid exchange is located in a position in sequence section (c-1);
the first amino acid exchange is located in a position in sequence section (b-1) and the second amino acid exchange is located in a position in sequence section (c-2);
the first amino acid exchange is located in a position in sequence section (b-2) and the second amino acid exchange is also located in a position in sequence section (b-2);
the first amino acid exchange is located in a position in sequence section (b-2) and the second amino acid exchange is located in a position in sequence section (c-1);
the first amino acid exchange is located in a position in sequence section (b-2) and the second amino acid exchange is located in a position in sequence section (c-2); or
the first amino acid exchange is located in a position in sequence section (c-1) and the second amino acid exchange is located in a position in sequence section (c-2).

In preferred embodiments, the amino acid sequence of the enzyme of the invention has at least two amino acid substitutions compared to SEQ ID NO. 2, of which
the first amino acid exchange is located in a position in sequence section (a-1) and the second amino acid exchange is located in a position in sequence section (a-1);
the first amino acid exchange is located in a position in sequence section (a-1) and the second amino acid exchange is located in a position in sequence section (b-1);
the first amino acid exchange is located in a position in sequence section (a-2) and the second amino acid exchange is located in a position in sequence section (b-1); or
the first amino acid exchange is located in a position in sequence section (b-2) and the second amino acid exchange is located in a position in sequence section (b-2).

In preferred embodiments, the amino acid sequence of the enzyme of the invention has at least three amino acid substitutions compared to SEQ ID NO. 2, of which
the first amino acid exchange is in a position in sequence portion (a-1), the second amino acid exchange is in a position in sequence portion (a-1), and the third amino acid exchange is in a position in sequence portion (a-1);
the first amino acid exchange is located in a position in sequence section (a-1), the second amino acid exchange is located in a position in sequence section (a-1), and the third amino acid exchange is located in a position in sequence section (a-2);
the first amino acid exchange is located in a position in sequence section (a-1), the second amino acid exchange is located in a position in sequence section (a-1), and the third amino acid exchange is located in a position in sequence section (a-3);
the first amino acid exchange is located in a position in sequence portion (a-1), the second amino acid exchange is located in a position in sequence portion (a-1), and the third amino acid exchange is located in a position in sequence portion (b-1);
the first amino acid exchange is located in a position in sequence portion (a-1), the second amino acid exchange is located in a position in sequence portion (a-1), and the third amino acid exchange is located in a position in sequence portion (b-2);
the first amino acid exchange is located in a position in sequence portion (a-1), the second amino acid exchange is located in a position in sequence portion (a-1), and the third amino acid exchange is located in a position in sequence portion (c-1);
the first amino acid exchange is located in a position in sequence portion (a-1), the second amino acid exchange is located in a position in sequence portion (a-1), and the third amino acid exchange is located in a position in sequence portion (c-2);
the first amino acid exchange is located in a position in sequence section (a-1), the second amino acid exchange is located in a position in sequence section (a-2), and the third amino acid exchange is located in a position in sequence section (a-3);
the first amino acid exchange is located in a position in sequence section (a-1), the second amino acid exchange is located in a position in sequence section (a-2), and the third amino acid exchange is located in a position in sequence section (b-1);
the first amino acid exchange is located in a position in sequence section (a-1), the second amino acid exchange is located in a position in sequence section (a-2), and the third amino acid exchange is located in a position in sequence section (b-2);
the first amino acid exchange is located in a position in sequence portion (a-1), the second amino acid exchange is located in a position in sequence portion (a-2), and the third amino acid exchange is located in a position in sequence portion (c-1);
the first amino acid exchange is located in a position in sequence portion (a-1), the second amino acid exchange is located in a position in sequence portion (a-2), and the third amino acid exchange is located in a position in sequence portion (c-2);
the first amino acid exchange is located in a position in sequence section (a-1), the second amino acid exchange is located in a position in sequence section (a-3), and the third amino acid exchange is located in a position in sequence section (b-1);
the first amino acid exchange is located in a position in sequence section (a-1), the second amino acid exchange is located in a position in sequence section (a-3), and the third amino acid exchange is located in a position in sequence section (b-2);
the first amino acid exchange is located in a position in sequence section (a-1), the second amino acid exchange is located in a position in sequence section (a-3), and the third amino acid exchange is located in a position in sequence section (c-1);
the first amino acid exchange is located in a position in sequence section (a-1), the second amino acid exchange is located in a position in sequence section (a-3), and the third amino acid exchange is located in a position in sequence section (c-2);
the first amino acid exchange is located in a position in sequence portion (a-1), the second amino acid exchange is located in a position in sequence portion (b-1), and the third amino acid exchange is located in a position in sequence portion (b-1);
the first amino acid exchange is located in a position in sequence portion (a-1), the second amino acid exchange is located in a position in sequence portion (b-1), and the third amino acid exchange is located in a position in sequence portion (b-2);
the first amino acid exchange is located in a position in sequence section (a-1), the second amino acid exchange is located in a position in sequence section (b-1), and the third amino acid exchange is located in a position in sequence section (c-1);
the first amino acid exchange is located in a position in sequence section (a-1), the second amino acid exchange is located in a position in sequence section (b-1), and the third amino acid exchange is located in a position in sequence section (c-2);
the first amino acid exchange is located in a position in sequence portion (a-1), the second amino acid exchange is located in a position in sequence portion (b-2), and the third amino acid exchange is located in a position in sequence portion (b-2);
the first amino acid exchange is located in a position in sequence section (a-1), the second amino acid exchange is located in a position in sequence section (b-2), and the third amino acid exchange is located in a position in sequence section (c-1);
the first amino acid exchange is located in a position in sequence portion (a-1), the second amino acid exchange is located in a position in sequence portion (b-2), and the third amino acid exchange is located in a position in sequence portion (b-2);
the first amino acid exchange is located in a position in sequence section (a-1), the second amino acid exchange is located in a position in sequence section (b-2), and the third amino acid exchange is located in a position in sequence section (c-2);
the first amino acid exchange is located in a position in sequence section (a-1), the second amino acid exchange is located in a position in sequence section (c-1), and the third amino acid exchange is located in a position in sequence section (c-2);
the first amino acid exchange is located in a position in sequence section (a-2), the second amino acid exchange is located in a position in sequence section (a-3), and the third amino acid exchange is located in a position in sequence section (b-1);
the first amino acid exchange is located in a position in sequence section (a-2), the second amino acid exchange is located in a position in sequence section (a-3), and the third amino acid exchange is located in a position in sequence section (b-2);
the first amino acid exchange is located in a position in sequence section (a-2), the second amino acid exchange is located in a position in sequence section (a-3), and the third amino acid exchange is located in a position in sequence section (c-1);
the first amino acid exchange is located in a position in sequence section (a-2), the second amino acid exchange is located in a position in sequence section (a-3), and the third amino acid exchange is located in a position in sequence section (c-2);
the first amino acid exchange is located in a position in sequence portion (a-2), the second amino acid exchange is located in a position in sequence portion (b-1), and the third amino acid exchange is located in a position in sequence portion (b-1);
the first amino acid exchange is located in a position in sequence section (a-2), the second amino acid exchange is located in a position in sequence section (b-1), and the third amino acid exchange is located in a position in sequence section (b-2);
the first amino acid exchange is located in a position in sequence section (a-2), the second amino acid exchange is located in a position in sequence section (b-1), and the third amino acid exchange is located in a position in sequence section (c-1);
the first amino acid exchange is located in a position in sequence section (a-2), the second amino acid exchange is located in a position in sequence section (b-1), and the third amino acid exchange is located in a position in sequence section (c-2);
the first amino acid exchange is located in a position in sequence portion (a-2), the second amino acid exchange is located in a position in sequence portion (b-2), and the third amino acid exchange is located in a position in sequence portion (b-2);
the first amino acid exchange is located in a position in sequence section (a-2), the second amino acid exchange is located in a position in sequence section (b-2), and the third amino acid exchange is located in a position in sequence section (c-1);
the first amino acid exchange is located in a position in sequence section (a-2), the second amino acid exchange is located in a position in sequence section (b-2), and the third amino acid exchange is located in a position in sequence section (c-2);
the first amino acid exchange is located in a position in sequence section (a-2), the second amino acid exchange is located in a position in sequence section (c-1), and the third amino acid exchange is located in a position in sequence section (c-2);
the first amino acid exchange is located in a position in sequence section (a-3), the second amino acid exchange is located in a position in sequence section (b-1), and the third amino acid exchange is located in a position in sequence section (b-1);
the first amino acid exchange is located in a position in sequence section (a-3), the second amino acid exchange is located in a position in sequence section (b-1), and the third amino acid exchange is located in a position in sequence section (b-2);
the first amino acid exchange is located in a position in sequence section (a-3), the second amino acid exchange is located in a position in sequence section (b-1), and the third amino acid exchange is located in a position in sequence section (c-1);
the first amino acid exchange is located in a position in sequence section (a-3), the second amino acid exchange is located in a position in sequence section (b-1), and the third amino acid exchange is located in a position in sequence section (c-2);
the first amino acid exchange is located in a position in sequence section (a-3), the second amino acid exchange is located in a position in sequence section (b-2), and the third amino acid exchange is located in a position in sequence section (b-2);
the first amino acid exchange is located in a position in sequence section (a-3), the second amino acid exchange is located in a position in sequence section (b-2), and the third amino acid exchange is located in a position in sequence section (c-1);
the first amino acid exchange is located in a position in sequence section (a-3), the second amino acid exchange is located in a position in sequence section (b-2), and the third amino acid exchange is located in a position in sequence section (c-2);
the first amino acid exchange is located in a position in sequence section (a-3), the second amino acid exchange is located in a position in sequence section (c-1), and the third amino acid exchange is located in a position in sequence section (c-2);
the first amino acid exchange is located in a position in sequence section (b-1), the second amino acid exchange is located in a position in sequence section (b-1), and the third amino acid exchange is located in a position in sequence section (b-2);
the first amino acid exchange is located in a position in sequence portion (b-1), the second amino acid exchange is located in a position in sequence portion (b-1), and the third amino acid exchange is located in a position in sequence portion (c-1);
the first amino acid exchange is located in a position in sequence section (b-1), the second amino acid exchange is located in a position in sequence section (b-1), and the third amino acid exchange is located in a position in sequence section (c-2);
the first amino acid exchange is located in a position in sequence section (b-1), the second amino acid exchange is located in a position in sequence section (b-2), and the third amino acid exchange is located in a position in sequence section (b-2);
the first amino acid exchange is located in a position in sequence portion (b-1), the second amino acid exchange is located in a position in sequence portion (b-2), and the third amino acid exchange is located in a position in sequence portion (c-1);
the first amino acid exchange is located in a position in sequence portion (b-1), the second amino acid exchange is located in a position in sequence portion (b-2), and the third amino acid exchange is located in a position in sequence portion (c-2);
the first amino acid exchange is located in a position in sequence section (b-1), the second amino acid exchange is located in a position in sequence section (c-1), and the third amino acid exchange is located in a position in sequence section (c-2);
the first amino acid exchange is located in a position in sequence portion (b-2), the second amino acid exchange is located in a position in sequence portion (b-2), and the third amino acid exchange is located in a position in sequence portion (c-1);
the first amino acid exchange is located in a position in sequence section (b-2), the second amino acid exchange is located in a position in sequence section (b-2), and the third amino acid exchange is located in a position in sequence section (c-2); or
the first amino acid exchange is located in a position in sequence portion (b-2), the second amino acid exchange is located in a position in sequence portion (c-1), and the third amino acid exchange is located in a position in sequence portion (c-2).

In preferred embodiments, the amino acid sequence of the enzyme of the invention has at least three amino acid substitutions compared to SEQ ID NO. 2, of which
the first amino acid exchange is located in a position in sequence section (a-1), the second amino acid exchange is located in a position in sequence section (a-1), and the third amino acid exchange is located in a position in sequence section (b-1); or
the first amino acid exchange is located in a position in sequence section (a-3), the second amino acid exchange is located in a position in sequence section (b-2), and the third amino acid exchange is located in a position in sequence section (b-2).

In a preferred embodiment of the invention, the amino acid sequence of the enzyme according to the invention has, compared to SEQ ID NO. 2, at least one, preferably two, more preferably three or more amino acid substitutions selected from the group consisting of positions S33, W41, D68, A74, V94, G175, Y201, T217, P221, S225, L229, V317, D328, L424, G445, M448, A453, P454, C457, and A507, preferably selected from the group consisting of positions S33, W41, D68, A74, V94, G175, Y201, S225, V317, L424, G445, M448, A453, and A507, more preferably selected from the group consisting of S33, W41, D68, A74, V94, Y201, S225, L424, G445, M448, A453, and A507, and most preferably selected from the group consisting of positions S33, W41, D68, A74, V94, Y201, S225, L424, G445, M448, A453.

In a preferred embodiment of the invention, the amino acid sequence of the enzyme of the invention has, compared to SEQ ID NO. 2, at least one, preferably two, more preferably three or more amino acid substitutions selected from one of the groups of positions
S33, D68, A74, G175, S225, L424, G445, A453, and A507;
S33, D68, A74, S225, L424, G445, A453, and A507;
S33, L424, G445, and A453;
S33, L424, and G445;
L424, and G445;
S33, D68, A74, and A507;
D68, A74, and A507; or
D68, and A74.

In a preferred embodiment of the invention, the amino acid sequence of the enzyme according to the invention has, compared to SEQ ID NO. 2, at least one, more, or all of the amino acid substitutions selected from positions S33, D68, A74, G175, S225, L424, G445, A453, and A507; preferably all of positions SS33, D68, A74, G175, S225, L424, G445, A453, and A507; S33, D68, A74, S225, L424, G445, A453, and A507; S33, L424, G445, and A453; S33, L424, and G445; L424, and G445; S33, D68, A74, and A507; D68, A74, and A507; or D68, and A74.

In preferred embodiments, the amino acid sequence of the enzyme of the invention, compared to SEQ ID NO. 2, does not have an amino acid exchange in at least one of the following positions: E24, S25, D26, L27, P28, A32, S33, T35, L37, L38, S40, W41, N42, K43, V44, E45, E46, Y48, A49, E50, V51, A52, P53, T54, Q57, S59, W60, T61, R62, P63, A65, E66, D67, D68, K69, A72, W73, V75, Q76, T77, S78, I79, T80, E81, T82, S83, E84, G85, P86, L87, A88, T91, V92, A93, V94, K95, P156, S170, S194, I330, N348, and E509 (cf. FIG. 1), further preferably does not have an amino acid exchange in at least one of the following positions: E24, S25, D26, L27, P28, A32, T35, L37, L38, S40, W41, N42, K43, V44, E45, E46, Y48, A49, E50, V51, A52, P53, T54, Q57, S59, W60, T61, R62, P63, A65, E66, D67, D68, K69, A72, W73, V75, Q76, T77, S78. I79, T80, E81, T82, S83, E84, G85, P86, L87, A88, T91, V92, A93, V94, K95, P156, S170, S194, I330, N348 and E509 (cf. FIG. 1), whereby in this case, in a particularly preferred embodiment, there is no substitution to alanine at position S33. The amino acid sequence according to the invention is different from SEQ ID NO. 3 according to WO 2004/083423 A1.

In preferred embodiments, the amino acid sequence of the enzyme of the invention has, compared to SEQ ID NO.2, an amino acid substitution at position G445. Preferably, the amino acid substitution is selected from G445A, G445V, G445L, G445I, G445M, G445P, G445F, G445W, G445Y, G445S, G445T, G445C, G445N, G445Q, G445D, G445E, G445R, G445K and G445H; preferably G445A, G445V, G445L, G445I,G445M, G445P, G445F, G445W, G445Y, G445S, G445T, G445C, G445N and G445Q; more preferably G445A and G445S.

In preferred embodiments, the amino acid sequence of the enzyme of the invention has, compared to SEQ ID NO. 2, an amino acid substitution at position A453. Preferably, the amino acid substitution is selected from A453G, A453V, A453L, A453I, A453M, A453P, A453F, A453W, A453Y, A453S, A453T, A453C, A453N, A453Q, A453D, A453E, A453R, A453K and A453H; preferably A453Y, A453S, A453T, A453C, A453N, A453Q, A453D, A453E, A453R, A453K and A453H; more preferably A453C, A453D, A453E, A453K, A453N, A453Q, A453R and A453S.

In preferred embodiments, the amino acid sequence of the enzyme of the invention has, compared to SEQ ID NO. 2, an amino acid substitution at position L424. Preferably, the amino acid substitution is selected from L424G, L424A, L424V, L424I, L424M, L424P, L424F, L424W, L424Y, L424S, L424T, L424C, L424N, L424Q, L424D, L424E, L424R, L424K and L424H; preferably L424G, L424A, L424V, L424I, L424M, L424P, L424F and L424W; more preferably L424V.

In preferred embodiments, the amino acid sequence of the enzyme of the invention has, compared to SEQ ID NO. 2, an amino acid substitution at position M448. Preferably, the amino acid substitution is selected from M448G, M448A, M448V, M448L, M448I, M448P, M448F, M448W, M448Y, M448S, M448T, M448C, M448N, M448Q, M448D, M448E, M448R, M448K and M448H; preferably M448D, M448E, M448R, M448K and M448H; preferably M448H.

In preferred embodiments, the amino acid sequence of the enzyme of the invention has, compared to SEQ ID NO. 2, an amino acid substitution at position A507. Preferably, the amino acid substitution is selected from A507G, A507V, A507L, A507I, A507M, A507P, A507F, A507W, A507Y, A507S, A507T, A507C, A507N, A507Q, A507D, A507E, A507R, A507K, A507H; preferably A507G, A507V, A507L, A507I, A507M, A507P, A507F and A507W; more preferably A507P.

In preferred embodiments, the amino acid sequence of the enzyme of the invention has, compared to SEQ ID NO.2, an amino acid substitution at position S33. Preferably, the amino acid substitution is selected from S33G, S33A, S33V, S33L, S33I, S33M, S33P, S33F, S33W, S33Y, S33S, S33T, S33C, S33N, S33Q, S33D, S33E, S33R, S33K, and S33H; preferably S33F, S33R, S33H, and S33Y.

In preferred embodiments, the amino acid sequence of the enzyme of the invention has, compared to SEQ ID NO. 2, an amino acid substitution at position W41. Preferably, the amino acid exchange is selected from W41G, W41A, W41V, W41L, W41I, W41M, W41P, W41F, W41Y, W41S, W41T, W41C, W41N, W41Q, W41D, W41E, WA1R, W41K, and W41H; preferably W41Y, W41S, W41T, W41C, W41N, and W41Q; more preferably W41Y.

In preferred embodiments, the amino acid sequence of the enzyme of the invention has, compared to SEQ ID NO. 2, an amino acid substitution at position D68. Preferably, the amino acid substitution is selected from D68G, D68A, D68V, D68L, D68I, D68M, D68P, D68F, D68W, D68Y, D68S, D68T, D68C, D68N, D68Q, D68E, D68R, D68K, and D68H; preferably D68Y, D68S, D68T, D68C, D68N, and D68Q; more preferably D68N.

In preferred embodiments, the amino acid sequence of the enzyme of the invention has, compared to SEQ ID NO.2, an amino acid substitution at position A74. Preferably, the amino acid substitution is selected from A74G, A74V, A74L, A74I, A74M, A74P, A74F, A74W, A74Y, A74S, A74T, A74C, A74N, A74Q, A74D, A74E, A74R, A74K, and A74H; preferably A74Y, A74S, A74T, A74C, A74N, and A74Q; more preferably A74Y.

In preferred embodiments, the amino acid sequence of the enzyme of the invention has, compared to SEQ ID NO.2, an amino acid substitution at position V94. Preferably, the amino acid substitution is selected from V94G, V94A, V94L, V94I, V94M, V94P, V94F, V94W, V94Y, V94S, V94T, V94C, V94N, V94Q, V94D, V94E, V94R, V94K, and V94H; preferably V94G, V94A, V94L, V94I,V94M, V94P, V94F, and V94W; more preferably V94I.

In preferred embodiments, the amino acid sequence of the enzyme of the invention has, compared to SEQ ID NO. 2, an amino acid substitution at position Y201. Preferably, the amino acid substitution is selected from Y201G, Y201A, Y201V, Y201L, Y201I, Y201M, Y201P, Y201F, Y201W, Y201S, Y201T, Y201C, Y201N, Y201Q, Y201D, Y201E, Y201R, Y201K and Y201H; preferably Y201G, Y201A, Y201V, Y201L, Y201I, Y201M, Y201P, Y201F and Y201W; more preferably Y201F.

In preferred embodiments, the amino acid sequence of the enzyme of the invention has, compared to SEQ ID NO. 2, an amino acid substitution in position P221. Preferably, the amino acid substitution is selected from P221G, P221A, P221V, P221L, P221I, P221M, P221F, P221W, P221Y, P221S, P221T, P221C, P221N, P221Q, P221D, P221E, P221R, P221K and P221H; preferably P221G, P221A, P221V, P221L, P221I, P221M, P221F, P221W, P221Y, P221S, P221T, P221C, P221N and P221Q; more preferably P221G and P221Q.

In preferred embodiments, the amino acid sequence of the enzyme of the invention has, compared to SEQ ID NO. 2, an amino acid substitution at position S225. Preferably, the amino acid substitution is selected from S225G, S225A, S225V, S225L, S225I, S225M, S225P, S225F, S225W, S225Y, S225T, S225C, S225N, S225Q, S225D, S225E, S225R, S225K, and S225H; preferably S225Y, S225T, S225C, S225N, and S225Q; more preferably S225T.

In preferred embodiments, the amino acid sequence of the enzyme of the invention has, compared to SEQ ID NO. 2, an amino acid substitution at position G175. Preferably, the amino acid substitution is selected from G175A, G175V, G175L, G175I, G175M, G175P, G175F, G175W, G175Y, G175S, G175T, G175C, G175N, G175Q, G175D, G175E, G175R, G175K and G175H; preferably G175A, G175V, G175L, G175I,G175M, G175P, G175F and G175W; more preferably G175A.

In preferred embodiments, the amino acid sequence of the enzyme of the invention has, compared to SEQ ID NO. 2, an amino acid substitution at position T217. Preferably, the amino acid exchange is selected from T217G, T217A, T217V, T217L, T217I, T217M, T217P, T217F, T217W, T217Y, T217S, T217C, T217N, T217Q, T217D, T217E, T217R, T217K and T217H; preferably T217D, T217E, T217R, T217K and T217H; more preferably T217R.

In preferred embodiments, the amino acid sequence of the enzyme of the invention has, compared to SEQ ID NO. 2, an amino acid substitution at position L229. Preferably, the amino acid substitution is selected from L229G, L229A, L229V, L229I, L229M, L229P, L229F, L229W, L229Y, L229S, L229T, L229C, L229N, L229Q, L229D, L229E, L229R, L229K and L229H; preferably L229Y, L229S, L229T, L229C, L229N and L229Q; more preferably L229C.

In preferred embodiments, the amino acid sequence of the enzyme of the invention has, compared to SEQ ID NO. 2, an amino acid substitution at position V317. Preferably, the amino acid substitution is selected from V317G, V317A, V317L, V317I, V317M, V317P, V317F, V317W, V317Y, V317S, V317T, V317C, V317N, V317Q, V317D, V317E, V317R, V317K and V317H; preferably V317G, V317A, V317L, V317I, V317M, V317P, V317F and V317W; more preferably V317I.

In preferred embodiments, the amino acid sequence of the enzyme of the invention has, compared to SEQ ID NO. 2, an amino acid substitution at position D328. Preferably, the amino acid substitution is selected from D328G, D328A, D328V, D328L, D328I, D328M, D328P, D328F, D328W, D328Y, D328S, D328T, D328C, D328N, D328Q, D328E, D328R, D328K and D328H; preferably D328E, D328R, D328K and D328H; more preferably D328R.

In preferred embodiments, the amino acid sequence of the enzyme of the invention has, compared to SEQ ID NO. 2, an amino acid substitution at position P454. Preferably, the amino acid substitution is selected from P454G, P454A, P454V, P454L, P454I, P454M, P454F, P454W, P454Y, P454S, P454T, P454C, P454N, P454Q, P454D, P454E, P454R, P454K and P454H; preferably P454Y, P454S, P454T, P454C, P454N and P454Q; more preferably P454N.

In preferred embodiments, the amino acid sequence of the enzyme of the invention has, compared to SEQ ID NO. 2, an amino acid substitution at position V457. Preferably, the amino acid substitution is selected from V457G, V457A, V457L, V457I, V457M, V457P, V457F, V457W, V457Y, V457S, V457T, V457C, V457N, V457Q, V457D, V457E, V457R, V457K and V457H; preferably V457G, V457A, V457L, V457I, V457M, V457P, V457F and V457W; more preferably V457G.

In preferred embodiments, the enzyme according to the invention has, compared to SEQ ID NO. 2, at least one amino acid exchange, preferably at least two amino acid exchanges, more preferably at least three amino acid exchanges, more preferably at least four amino acid exchanges, more preferably at least five amino acid exchanges, more preferably at least six amino acid exchanges, more preferably at least seven amino acid exchanges, more preferably at least eight amino acid exchanges, more preferably at least nine amino acid exchanges, still more preferably at least ten amino acid exchanges, and most preferably at least eleven amino acid exchanges which are independently selected from
G445A, G445V, G445L, G445I,G445M, G445P, G445F, G445W, G445Y, G445S, G445T, G445C, G445N, G445Q, G445D, G445E, G445R, G445K and G445H; preferred G445A, G445V, G445L, G445I,G445M, G445P, G445F, G445W, G445Y, G445S, G445T, G445C, G445N and G445Q; preferably G445A and G445S;
A453G, A453V, A453L, A453I, A453M, A453P, A453F, A453W, A453Y, A453S, A453T, A453C, A453N, A453Q, A453D, A453E, A453R, A453K and A453H; preferably A453Y, A453S, A453T, A453C, A453N, A453Q, A453D, A453E, A453R, A453K and A453H; more preferably A453C, A453D, A453E, A453K, A453N, A453Q, A453R and A453S;
L424G, L424A, L424V, L424I, L424M, L424P, L424F, L424W, L424Y, L424S, L424T, L424C, L424N, L424Q, L424D, L424E, L424R, L424K and L424H; preferably L424G, L424A, L424V, L424I, L424M, L424P, L424F and L424W; more preferably L424V;
M448G, M448A, M448V, M448L, M448I, M448P, M448F, M448W, M448Y, M448S, M448T, M448C, M448N, M448Q, M448D, M448E, M448R, M448K and M448H; preferably M448D, M448E, M448R, M448K and M448H; more preferably M448H;
A507G, A507V, A507L, A507I, A507M, A507P, A507F, A507W, A507Y, A507S, A507T, A507C, A507N, A507Q, A507D, A507E, A507R, A507K, A507H; preferably A507G, A507V, A507L, A507I, A507M, A507P, A507F and A507W; more preferably A507P;
P454G, P454A, P454V, P454L,P454I, P454M, P454F, P454W, P454Y, P454S, P454T, P454C, P454N, P454Q, P454D, P454E, P454R, P454K and P454H; preferably P454Y, P454S, P454T, P454C, P454N and P454Q; more preferably P454N;
V457G, V457A, V457L, V457I, V457M, V457P, V457F, V457W, V457Y, V457S, V457T, V457C, V457N, V457Q, V457D, V457E, V457R, V457K and V457H; preferably V457G, V457A, V457L, V457I, V457M, V457P, V457F and V457W; more preferably V457G;
S33G, S33A, S33V, S33L, S33I, S33M, S33P, S33F, S33W, S33Y, S33S, S33T, S33C, S33N, S33Q, S33D, S33E, S33R, S33K, and S33H; preferably S33F, S33R, S33H, and S33Y;
W41G, W41A, W41V, W41L, W41I, W41M, W41P, W41F, W41Y, W41S, W41T, W41C, W41N, W41Q, W41D, W41E, W41R, W41K, and W41H; preferably W41Y, W41S, W41T, W41C, W41N, and W41Q; more preferably W41Y;
D68G, D68A, D68V, D68L, D68I, D68M, D68P, D68F, D68W, D68Y, D68S, D68T, D68C, D68N, D68Q, D68E, D68R, D68K and D68H; preferably D68Y, D68S, D68T, D68C, D68N and D68Q; more preferably D68N;
A74G, A74V, A74L, A74I, A74M, A74P, A74F, A74W, A74Y, A74S, A74T, A74C, A74N, A74Q, A74D, A74E, A74R, A74K and A74H; preferably A74Y, A74S, A74T, A74C, A74N and A74Q; more preferably A74Y;
V94G, V94A, V94L, V94I,V94M, V94P, V94F, V94W, V94Y, V94S, V94T, V94C, V94N, V94Q, V94D, V94E, V94R, V94K and V94H; preferably V94G, V94A, V94L, V94I,V94M, V94P, V94F and V94W; more preferably V94I;
V317G, V317A, V317L, V317I, V317M, V317P, V317F, V317W, V317Y, V317S, V317T, V317C, V317N, V317Q, V317D, V317E, V317R, V317K and V317H; preferably V317G, V317A, V317L, V317I, V317M, V317P, V317F and V317W; more preferably V317I;
D328G, D328A, D328V, D328L, D328I, D328M, D328P, D328F, D328W, D328Y, D328S, D328T, D328C, D328N, D328Q, D328E, D328R, D328K and D328H; preferably D328E, D328R, D328K and D328H; more preferably D328R;
Y201G, Y201A, Y201V, Y201L, Y201I, Y201M, Y201P, Y201F, Y201W, Y201S, Y201T, Y201C, Y201N, Y201Q, Y201D, Y201E, Y201R, Y201K, and Y201H; preferably Y201G, Y201A, Y201V, Y201L, Y201I, Y201M, Y201P, Y201F, and Y201W; more preferably Y201F; and
S225G, S225A, S225V, S225L, S225I, S225M, S225P, S225F, S225W, S225Y, S225T, S225C, S225N, S225Q, S225D, S225E, S225R, S225K and S225H; preferably S225Y, S225T, S225C, S225N and S225Q; more preferably S225T;
G175A, G175V, G175L, G175I,G175M, G175P, G175F, G175W, G175Y, G175S, G175T, G175C, G175N, G175Q, G175D, G175E, G175R, G175K and G175H; preferably G175A, G175V, G175L, G175I,G175M, G175P, G175F and G175W; more preferably G175A;
T217G, T217A, T217V, T217L, T217I, T217M, T217P, T217F, T217W, T217Y, T217S, T217C, T217N, T217Q, T217D, T217E, T217R, T217K and T217H; preferably T217D, T217E, T217R, T217K and T217H; more preferably T217R;
P221G, P221A, P221V, P221L, P221I, P221M, P221F, P221W, P221Y, P221S, P221T, P221C, P221N, P221Q, P221D, P221E, P221R, P221K and P221H; preferably P221G, P221A, P221V, P221L, P221I, P221M, P221F, P221W, P221Y, P221S, P221T, P221C, P221N and P221Q; more preferably P221G;
L229G, L229A, L229V, L229I, L229M, L229P, L229F, L229W, L229Y, L229S, L229T, L229C, L229N, L229Q, L229D, L229E, L229R, L229K and L229H; preferably L229Y, L229S, L229T, L229C, L229N and L229Q; more preferably L229C.

In preferred embodiments, the enzyme according to the invention has, compared to SEQ ID NO. 2, at least one amino acid exchange, preferably at least two amino acid exchanges, more preferably at least three amino acid exchanges, more preferably at least four amino acid exchanges, more preferably at least five amino acid exchanges, more preferably at least six amino acid exchanges, more preferably at least seven amino acid exchanges, more preferably at least eight amino acid exchanges, more preferably at least nine amino acid exchanges, still more preferably at least ten amino acid exchanges, and most preferably at least eleven amino acid exchanges which are independently selected from
G445A, G445V, G445L, G445I,G445M, G445P, G445F, G445W, G445Y, G445S, G445T, G445C, G445N, G445Q, G445D, G445E, G445R, G445K and G445H; preferably G445A, G445V, G445L, G445I,G445M, G445P, G445F, G445W, G445Y, G445S, G445T, G445C, G445N and G445Q; more preferably G445A and G445S;
A453G, A453V, A453L, A453I, A453M, A453P, A453F, A453W, A453Y, A453S, A453T, A453C, A453N, A453Q, A453D, A453E, A453R, A453K and A453H; preferably A453Y, A453S, A453T, A453C, A453N, A453Q, A453D, A453E, A453R, A453K and A453H; more preferably A453C, A453D, A453E, A453K, A453N, A453Q, A453R and A453S;
L424G, L424A, L424V, L424I, L424M, L424P, L424F, L424W, L424Y, L424S, L424T, L424C, L424N, L424Q, L424D, L424E, L424R, L424K and L424H; preferably L424G, L424A, L424V, L424I, L424M, L424P, L424F and L424W; more preferably L424V;
M448G, M448A, M448V, M448L, M448I, M448P, M448F, M448W, M448Y, M448S, M448T, M448C, M448N, M448Q, M448D, M448E, M448R, M448K and M448H; preferably M448D, M448E, M448R, M448K and M448H; more preferably M448H;
A507G, A507V, A507L, A507I, A507M, A507P, A507F, A507W, A507Y, A507S, A507T, A507C, A507N, A507Q, A507D, A507E, A507R, A507K, A507H; preferably A507G, A507V, A507L, A507I, A507M, A507P, A507F and A507W, more preferably A507P;
S33G, S33A, S33V, S33L, S33I, S33M, S33P, S33F, S33W, S33Y, S33S, S33T, S33C, S33N, S33Q, S33D, S33E, S33R, S33K, and S33H; preferably S33F, S33R, S33H, and S33Y;
W41G, W41A, W41V, W41L, W41I, W41M, W41P, W41F, W41Y, W41S, W41T, W41C, W41N, W41Q, W41D, W41E, W41R, W41K, and W41H; preferably W41Y, W41S, W41T, W41C, W41N, and W41Q; more preferably W41Y;
D68G, D68A, D68V, D68L, D68I, D68M, D68P, D68F, D68W, D68Y, D68S, D68T, D68C, D68N, D68Q, D68E, D68R, D68K and D68H; preferably D68Y, D68S, D68T, D68C, D68N and D68Q; more preferably D68N;
A74G, A74V, A74L, A74I, A74M, A74P, A74F, A74W, A74Y, A74S, A74T, A74C, A74N, A74Q, A74D, A74E, A74R, A74K and A74H; preferably A74Y, A74S, A74T, A74C, A74N and A74Q; more preferably A74Y;
V94G, V94A, V94L, V94I,V94M, V94P, V94F, V94W, V94Y, V94S, V94T, V94C, V94N, V94Q, V94D, V94E, V94R, V94K and V94H; preferably V94G, V94A, V94L, V94I,V94M, V94P, V94F and V94W; more preferably V94I;
Y201G, Y201A, Y201V, Y201L, Y201I, Y201M, Y201P, Y201F, Y201W, Y201S, Y201T, Y201C, Y201N, Y201Q, Y201D, Y201E, Y201R, Y201K, and Y201H; preferably Y201G, Y201A, Y201V, Y201L, Y201I, Y201M, Y201P, Y201F, and Y201W; more preferably Y201F; and
S225G, S225A, S225V, S225L, S225I, S225M, S225P, S225F, S225W, S225Y, S225T, S225C, S225N, S225Q, S225D, S225E, S225R, S225K and S225H; preferably S225Y, S225T, S225C, S225N and S225Q; more preferably S225T.

In preferred embodiments, the amino acid sequence of the enzyme of the invention has, compared to SEQ ID NO. 2, at least two amino acid substitutions in positions
S33 and W41; S33 and D68; S33 and A74; S33 and V94; S33 and Y201; S33 and S225; S33 and L424; S33 and G445; S33 and M448; S33 and A453; or S33 and A507;
W41 and D68; W41 and A74; W41 and V94; W41 and Y201; W41 and S225; W41 and L424; W41 and G445; W41 and M448; W41 and A453; or W41 and A507;
D68 and A74; D68 and V94; D68 and Y201; D68 and S225; D68 and L424; D68 and G445; D68 and M448; D68 and A453; or D68 and A507;
A74 and V94; A74 and Y201; A74 and S225; A74 and L424; A74 and G445; A74 and M448; A74 and A453; or A74 and A507;
V94 and Y201; V94 and S225; V94 and L424; V94 and G445; V94 and M448; V94 and A453; or V94 and A507;
Y201 and S225; Y201 and L424; Y201 and G445; Y201 and M448; or Y201 and A453;
S225 and L424; S225 and G445; S225 and M448; S225 and A453; or S225 and A507;
L424 and G445; L424 and M448; L424 and A453; or L424 and A507;
G445 and M448; G445 and A453; or G445 and A507;
M448 and A453; or M448 and A507; or
A453 and A507.

In preferred embodiments, the amino acid sequence of the enzyme of the invention has, compared to SEQ ID NO. 2, at least two amino acid substitutions, namely
in position G445 and in position S33;
in position G445 and in position D68;
in position G445 and in position A74;
in position G445 and in position S225;
in position S33 and in position D68;
in position S33 and in position A74;
in position S33 and in position S225;
in position S33 and in position L424;
in position S33 and in position M448;
in position D68 and in position A74;
in position D68 and in position S225;
in position D68 and in position A507;
in position A74 and in position S225;
in position A74 and in position A507;
in position L424 and in position M448.

In preferred embodiments, the amino acid sequence of the enzyme of the invention has, compared to SEQ ID NO. 2, at least three amino acid substitutions in positions
S33, W41 and D68; S33, W41 and A74; S33, W41 and V94; S33, W41 and Y201; S33, W41 and S225; S33, W41 and L424; S33, W41 and G445; S33, W41 and M448; S33, W41 and A453; S33, W41 and A507;
S33, D68 and A74; S33, D68 and V94; S33, D68 and Y201; S33, D68 and S225; S33, D68 and L424; S33, D68 and G445; S33, D68 and M448; S33, D68 and A453; S33, D68 and A507;
S33, A74 and V94; S33, A74 and Y201; S33, A74 and S225; S33, A74 and L424; S33, A74 and G445; S33, A74 and M448; S33, A74 and A453; S33, A74 and A507;
S33, V94 and Y201; S33, V94 and S225; S33, V94 and L424; S33, V94 and G445; S33, V94 and M448; S33, V94 and A453; S33, V94 and A507;
S33, Y201 and S225; S33, Y201 and L424; S33, Y201 and G445; S33, Y201 and M448; S33, Y201 and A453; S33, Y201 and A507;
S33, S225 and L424; S33, S225 and G445; S33, S225 and M448; S33, S225 and A453; S33, S225 and A507;
S33, L424 and G445; S33, L424 and M448; S33, L424 and A453; S33, L424 and A507;
S33, G445 and M448; S33, G445 and A453; S33, G445 and A507;
S33, M448, and A453; S33, M448, and A507;
S33, A453 and A507;
W41, D68 and A74; W41, D68 and V94; W41, D68 and Y201; W41, D68 and S225; W41, D68 and L424; W41, D68 and G445; W41, D68 and M448; W41, D68 and A453; W41, D68 and A507;
W41, A74 and V94; W41, A74 and Y201; W41, A74 and S225; W41, A74 and L424; W41, A74 and G445; W41, A74 and M448; W41, A74 and A453; W41, A74 and A507;
W41, V94 and Y201; W41, V94 and S225; W41, V94 and L424; W41, V94 and G445; W41, V94 and M448; W41, V94 and A453; W41, V94 and A507;
W41, Y201 and S225; W41, Y201 and L424; W41, Y201 and G445; W41, Y201 and M448; W41, Y201 and A453; W41, Y201 and A507;
W41, S225 and L424; W41, S225 and G445; W41, S225 and M448; W41, S225 and A453; A507;
W41, L424 and G445; W41, L424 and M448; W41, L424 and A453; W41, L424 and A507;
W41, G445 and M448; W41, G445 and A453; W41, G445 and A507;
W41, M448 and A453; W41, M448 and A507;
W41, A453 and A507;
D68, A74 and V94; D68, A74 and Y201; D68, A74 and S225; D68, A74 and L424; D68, A74 and G445; D68, A74 and M448; D68, A74 and A453; D68, A74 and A507;
D68, V94 and Y201; D68, V94 and S225; D68, V94 and L424; D68, V94 and G445; D68, V94 and M448; D68, V94 and A453; D68, V94 and A507;
D68, Y201 and S225; D68, Y201 and L424; D68, Y201 and G445; D68, Y201 and M448; D68, Y201 and A453; D68, Y201 and A507;
D68, S225 and L424; D68, S225 and G445; D68, S225 and M448; D68, S225 and A453; D68, S225 and A507;
D68, L424 and G445; D68, L424 and M448; D68, L424 and A453; D68, L424 and A507;
D68, G445 and M448; D68, G445 and A453; D68, G445 and A507;
D68, M448 and A453; D68, M448 and A507;
D68, A453 and A507;
A74, V94 and Y201; A74, V94 and S225; A74, V94 and L424; A74, V94 and G445; A74, V94 and M448; A74, V94 and A453; A74, V94 and A507;
A74, Y201 and S225; A74, Y201 and L424; A74, Y201 and G445; A74, Y201 and M448; A74, Y201 and A453; A74, Y201 and A507;
A74, S225 and L424; A74, S225 and G445; A74, S225 and M448; A74, S225 and A453; A74, S225 and A507;
A74, L424 and G445; A74, L424 and M448; A74, L424 and A453; A74, L424 and A507;
A74, G445 and M448; A74, G445 and A453; A74, G445 and A507;
A74, M448 and A453; A74, M448 and A507;
A74, A453 and A507;
V94, Y201 and S225; V94, Y201 and L424; V94, Y201 and G445; V94, Y201 and M448; V94, Y201 and A453; V94, Y201 and A507;
V94, S225 and L424; V94, S225 and G445; V94, S225 and M448; V94, S225 and A453; V94, S225 and A507;
V94, L424 and G445; V94, L424 and M448; V94, L424 and A453; V94, L424 and A507;
V94, G445 and M448; V94, G445 and A453; V94, G445 and A507;
V94, M448 and A453; V94, M448 and A507;
V94, A453 and A507;
Y201, S225 and L424; Y201, S225 and G445; Y201, S225 and M448; Y201, S225 and A453; Y201, S225 and A507;
Y201, L424 and G445; Y201, L424 and M448; Y201, L424 and A453; Y201, L424 and A507;
Y201, G445 and M448; Y201, G445 and A453; Y201, G445 and A507;
Y201, M448 and A453; Y201, M448 and A507;
Y201, A453 and A507;
S225, L424 and G445; S225, L424 and M448; S225, L424 and A453; S225, L424 and A507;
S225, G445 and M448; S225, G445 and A453; S225, G445 and A507;
S225, M448 and A453; S225, M448 and A507;
S225, A453 and A507;
L424, G445 and M448; L424, G445 and A453; L424, G445 and A507;
L424, M448 and A453; L424, M448 and A507;
L424, A453 and A507;
G445, M448 and A453; G445, M448 and A507;
G445, A453 and A507; or
M448, A453 and A507.

In preferred embodiments, the amino acid sequence of the enzyme of the invention has, compared to SEQ ID NO.2, at least three amino acid substitutions in positions
S33, G448 and A453; or
D68, A74, and A507.

In preferred embodiments, the amino acid sequence of the enzyme of the invention has, compared to SEQ ID NO. 2, at least two amino acid exchanges, preferably at least three amino acid exchanges, more preferably at least four amino acid exchanges, and most preferably all five amino acid exchanges selected from positions S33, D68, A74, S225, L424, G445 and A453.

In preferred embodiments, the amino acid sequence of the enzyme of the invention has, compared to SEQ ID NO. 2, a sequence identity of at least 86.0%, a sequence identity of at least 87%, a sequence identity of at least 88%, preferably at least 89%, more preferably at least 90%, more preferably at least 92%, even more preferably at least 94%, most preferably at least 96%, and especially at least 98%.

In a preferred embodiment of this aspect of the invention and its preceding embodiments, the amino acid sequence of the enzyme of the invention additionally has, compared to SEQ ID NO.2, at least one amino acid substitution selected from the group consisting of positions V94I,V317I, and D328R.

In preferred embodiments, the amino acid sequence of the enzyme according to the invention has a sequence identity of at least 95%, preferably at least 96%, more preferably at least 97%, most preferably at least 98% and in particular at least 99% to SEQ ID NO. 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46. 47, 48, 49, 50, 51, 52, 53, 54 or 55.

Although reference was already made in WO 2006/040345 to the use of other enzymes (not according to the invention) in food production processes, it was not possible there to demonstrate their effectiveness in the production of acrylamide-reduced products. Furthermore, with the enzyme according to the invention, it is possible to efficiently and rapidly degrade acrylamide in a preparation. This is particularly advantageous in continuous or semi-continuous large-scale processes such as in the food and luxury food industry, since this ensures short residence times in the enzyme treatment as well as rapid further processing of any perishable semi-finished goods. Very preferably and advantageously, the enzyme according to the invention can be used in coffee product/coffee substitute production, since the degradation of acrylamide is an essential process to obtain a safe and harmless final food product.

According to a preferred embodiment of the present invention, the enzyme is an amidase. Amidases or amidohydrolases are a class of enzymes that catalyze the hydrolysis of amide bonds. Amidases occur in large numbers in nature and are used in conventional products such as detergents and household cleaners. It has been shown that the use of at least one amidase is advantageous in the context of the present invention, as it cleaves the amide bond of acrylamide by a very simple mechanism, which does not require any additional agents or co-factors.

In a further preferred embodiment, the enzyme is suitable to be catalytically active up to a temperature of 50° C. or more, preferably at least up to a temperature of 60° C., more preferably at least up to a temperature of 70° C., further preferably at least up to a temperature of 80° C., and/or is used in the context of the present invention at such a temperature. In the context of the present invention, catalytic activity means that a detectable cleavage of the amide bond of the acrylamide occurs. Activity at temperatures up to 80° C. is particularly preferred in the context of the present invention, since most proteins and enzymes which are not thermotolerant lose their activity at a temperature above 42° C. With the aid of an enzyme according to the invention, it is advantageously possible to obtain catalytic activity of the enzyme even at temperatures up to 80° C. Such a high temperature is required for numerous processes in the food and luxury food sector, since important cooking, scalding and processing processes take place at temperatures above 50° C., in some cases up to 80° C. or more.

In yet another preferred embodiment, the enzyme is suitable to exhibit catalytic activity in a range of pH 4 to pH 7 and/or is used in the context of the present invention in such a pH range. According to a further preferred embodiment, the enzyme exhibits catalytic activity (at least) in a range from pH 4 to pH 6.5, preferably in a range from pH 4.5 to pH 5.5. Of course, the enzyme can also exhibit catalytic activity outside of these pH ranges or can be used in such ranges within the scope of the present invention. The pH plays a crucial role in the stability and activity of enzymes. A pH above or below the optimum pH usually results in partial to complete loss of activity. It is therefore all the more surprising that an enzyme according to the invention can be used efficiently in an acrylamide degradation step in preparations with a slightly acidic pH.

In one embodiment, the enzyme exhibits catalytic activity for at least 24 hours, preferably at least 48 hours, more preferably at least 72 hours, up to a temperature of 50° C. or more, preferably at least up to a temperature of 60° C., more preferably at least up to a temperature of 70° C., more preferably at least up to a temperature of 80° C., (at least) in a range from pH 4 to pH 7, preferably in a range from pH 4 to pH 6.5, more preferably in a range from pH 4.5 to pH 5.5.

In a further preferred embodiment, the enzyme exhibits catalytic activity up to a temperature of 50° C. or more, preferably at least up to a temperature of 60° C., more preferably at least up to a temperature of 70° C., further preferably at least up to a temperature of 80° C., (at least) in a range from pH 4 to pH 7, preferably in a range from pH 4 to pH 6.5, more preferably in a range from pH 4.5 to pH 5.5. Of course, the enzyme can also exhibit catalytic activity outside these pH ranges up to the temperatures mentioned and can accordingly be used in such ranges.

In connection with the investigations underlying the present invention, it was surprisingly possible to identify enzymes whose catalytic activity is also present or retained in an acidic pH range and at high temperatures. The use of such an enzyme is quite particularly advantageous in its use for acrylamide degradation in coffee/coffee substitute product manufacture, e.g. for the treatment of extracts from roasted coffee/coffee substitute products. After extraction, such preparations have a temperature above 50° C., often even above 70° C. or even above 80° C. It is well known that such extracts have a slightly acidic pH. Naturally occurring enzymes often do not exhibit sufficient catalytic activity under these conditions. It is therefore all the more advantageous to use enzymes according to the invention in such preparations in order to efficiently degrade acrylamide under these conditions.

According to a preferred embodiment of the present invention, the enzyme comprises or consists of an amino acid sequence having a sequence identity of at least 90%, 91%, 92%, 93%, 94%, 95%, 96%, 97%, 98% or 99% to a sequence selected from the group consisting of the sequences according to SEQ ID NO. 3 to SEQ ID NO. 55, preferably from the sequences according to SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21 and SEQ ID NO. 22, particularly preferably from the sequences according to SEQ ID NO. 19, SEQ ID NO. 20, SEQ ID NO. 21 and SEQ ID NO. 22.

In another preferred embodiment of the present invention, the enzyme is a modified amidase from Pseudonocardia thermophila. The organism Pseudonocardia thermophila is characterized by its distribution in rather hotter temperature environments, such as dung heaps, warm springs or the like. The organism belongs to the thermotolerant prokaryotes; it can grow at temperatures between 40 and 50° C. Due to its adaptation to warmer environments, its enzymes are also more thermotolerant, but starting from the wild type, not above 50° C. It is surprising that tolerance in the acidic pH range could be achieved starting from an enzyme of the organism Pseudonocardia thermophila, since Pseudonocardia thermophila is not acid tolerant by nature.

Another aspect of the present invention relates to a method for degrading acrylamide, preferably for reducing the amount of acrylamide in a preparation, consisting of or comprising the steps:
(i) Providing an enzyme of the invention (as described herein, preferably as described herein as preferred or particularly advantageous),
(ii) providing a mixture, preferably a preparation, containing acrylamide and adding the enzyme of step (i),
(iii) Incubating the mixture resulting from step (ii) for at least 20 minutes, preferably at a temperature in the range of 40° C. to 80° C., more preferably at a temperature in the range of 45° C. to 75° C.,
(iv) optionally, heating the incubated mixture resulting from step (iii) so that inactivation of the enzyme occurs, preferably by heating to a temperature of at least 90° C. and maintaining a temperature above 90° C. for at least 15 minutes, optionally cooling the mixture,
to obtain a product having a lower acrylamide content than the mixture or preparation provided in step (ii).

Incubation in the sense of the present invention means that the provided mixture from step (ii) remains at a certain temperature for a predetermined time. Holding the temperature in the sense of the present invention means that the temperature of the incubated mixture in step (iii) is not or not significantly changed for a defined duration. Minor fluctuations in the temperature are acceptable in this respect and can be assessed by the person skilled in the art.

In one embodiment of the present invention, the enzyme can be produced recombinantly, with suitable expression organisms and conditions being familiar to those skilled in the art. Furthermore, the enzyme may be present unpurified as a lysate, partially purified or highly purified. Suitable purification processes are known to the skilled person.

In a further embodiment, the enzyme according to the invention may be present as a solution or immobilized. Suitable immobilization processes are sufficiently known to the skilled person.

Inactivation refers to the loss of activity of the enzyme caused by an extremely high temperature as well as the unfolding of the amino acid chain of the enzyme. In yet another embodiment, the inactivated enzyme can then be removed from the preparation by methods commonly used by those skilled in the art, such as filtration, absorption or adsorption.

In one embodiment of the method of the present invention, the acrylamide contained in the initially provided mixture or preparation is the product of a Maillard reaction. A Maillard reaction can be observed, for example, during deep-frying or frying of food and manifests itself in a typical browning. The Maillard reaction is also essential in the roasting of coffee products to obtain the typical roasted taste. The product of the Maillard reaction is acrylamide, which is cleaved in the course of the process of the invention using the enzyme of the invention.

According to another preferred embodiment of the method of the present invention, the contained acrylamide of the provided preparation is the product of a Maillard reaction and the preparation is a preparation serving pleasure or nourishment or a cosmetic preparation, or a semi-finished product for the preparation of such preparations, preferably wherein the preparation is selected from the group consisting of fried or deep-fried potato products, roasted cereals or products containing them, com products, coffee products, e.g. solid or liquid coffee extracts and green coffee, chicory extracts, cereal coffee products, coffee substitutes, snacks, wheat products, cosmetics, bakery products or baked goods. e.g., biskuits, cookies, rusks, cereal bars, scones, ice cream cones, waffles, crumpets, gingerbread, crispbread and bread substitute products, pasta, rice, fish products, meat products, cereals, beer, nuts, complementary foods for children and infants, hair styling products, personal care products, hair care products, and facial care products.

The difference between semi-finished and finished goods lies in their degree of processing. Semi-finished goods are all products that are subjected to a further processing step. These can be, for example, roasted coffee extracts, doughs, green coffee, potato products, etc. Finished goods, on the other hand, are not processed further, but are packaged in their form and delivered to the consumer. Examples of finished goods include instant coffee, ready-to-use coffee powder, chips, noodles and the like.

According to a further preferred embodiment of the method of the present invention, the acrylamide content in the product obtained is < 2000 µg/kg, preferably < 850 µg/kg, particularly preferably < 500 µg/kg, in each case based on the total weight of the product. Whenever reference is made in the present disclosure to a value less than (“<”), this range of values — as far as possible and useful — preferably also includes 0. For example, the indication < 2000 µg/kg is a range of values from 0 to 2000 µg/kg.

In a further embodiment of the method according to the invention, the acrylamide content can be or is reduced by 60%, preferably by 65%, particularly preferably by 70%, further preferably by 75%, particularly preferably by 80%, further preferably by 85%, particularly preferably by 90%, further preferably by 95% and very particularly preferably by 100%, compared to a preparation that has not been subjected to the process according to the invention.

Another aspect of the present invention relates to a method for preparing a preparation serving pleasure or nourishment or a cosmetic preparation with reduced acrylamide content, preferably wherein the preparation is selected from the group consisting of fried or deep-fried potato products, roasted cereals or products containing them, com products, coffee products, e.g. solid or liquid coffee extracts and green coffee, chicory extracts, cereal coffee products, coffee substitute products, snacks, wheat products, cosmetics, baked goods or pastries, e.g. biscuits, cookies, rusks, cereal bars, scones, ice cream cones, waffles, crumpets, gingerbread, crispbread and bread substitute products, pasta, rice, fish products, meat products, cereals, beer, nuts, complementary foods for children and infants, hair styling products, personal care products, hair care products, and facial care products, which consists of or includes the following steps:
(I) (a) providing a product obtained by a process according to the invention (as described herein, preferably as described herein as preferred or particularly advantageous), and
(b) Further processing of the product and/or addition of one or more further ingredients to obtain the preparation serving pleasure or nourishment or the cosmetic preparation, or
(II) (i) providing an enzyme of the invention (as described herein, preferably as described herein as preferred or particularly advantageous),
   (ii) providing a preparation serving pleasure or nourishment or cosmetic preparation containing acrylamide and adding the enzyme of the invention from step (i),
   (iii) incubating the preparation resulting from step (ii) for at least 20 minutes, preferably at a temperature in the range of 40° C. to 80° C., more preferably at a temperature in the range of 45° C. to 75° C.,
   (iv) optionally heating the incubated preparation resulting from step (iii) so that inactivation of the enzyme occurs, preferably by heating to a temperature of at least 90° C. and maintaining a temperature above 90° C. for at least 15 minutes, optionally cooling the incubated preparation to obtain a preparation having a lower acrylamide content than the preparation provided in step (ii). In one embodiment of the method according to the invention, a semi-finished product whose acrylamide content has been reduced is further processed into a final product to obtain a preparation serving pleasure or nourishment or a cosmetic preparation. In a further embodiment, a semi-finished product is treated with an enzyme according to the invention to obtain a preparation with a reduced acrylamide content. This preparation can then be heated to above 90° C. to inactivate the enzyme. In yet another embodiment, the inactivated enzyme can then be removed from the preparation by methods commonly used by those skilled in the art, such as filtration, absorption or adsorption.

According to a further aspect, the present invention relates to the use of an enzyme according to the invention for the degradation of acrylamide and/or for the preparation of a preparation serving pleasure or nourishment or a cosmetic preparation having a reduced acrylamide content, preferably of < 2000 µg/kg, preferably < 850 µg/kg, particularly preferably < 500 µg/kg, in each case based on the total weight of the preparation. In a further embodiment, the acrylamide content can be or is reduced by 60%, preferably by 65%, particularly preferably by 70%, further preferably by 75%, particularly preferably by 80%, further preferably by 85%, particularly preferably by 90%, further preferably by 95% and very particularly preferably by 100% compared to a preparation in which the enzyme according to the invention was not used.

A further aspect of the present invention relates to a preparation serving pleasure or nourishment or a cosmetic preparation (preferably those as described above), prepared or preparable by a process according to the invention, wherein the acrylamide content is < 2000 µg/kg, preferably < 850 µg/kg, particularly preferably < 500 µg/kg (and/or wherein the acrylamide content is/are reduced as described above, preferably as described above as preferred), in each case based on the total weight of the preparation.

Preferred embodiments of the invention are summarized below as sentences 1 to 18:
Sentence 1. An enzyme for reducing the amount of acrylamide in a preparation comprising or consisting of an amino acid sequence having a sequence identity of at least 90%, 91%, 92%, 93%, 94%, 95%, 96%, 97%, 98% or 99% to a sequence according to SEQ ID NO. 1 or having a sequence according to SEQ ID NO. 1, wherein the amino acid sequence is not a sequence according to SEQ ID NO. 2.
Sentence 2. The enzyme of sentence 1, wherein the amino acid sequence has, at least at one, more or all of the positions selected from the group consisting of positions 33, 41, 68, 74, 94, 175, 201, 217, 221, 225, 229, 317, 328, 424, 445, 448, 453, 454, 457 and 507, an amino acid that does not correspond to the amino acid of the corresponding position(s) of the sequence according to SEQ ID NO. 2.
Sentence 3. The enzyme according to sentence 1 or 2, wherein the amino acid sequence has, at least at one, more or all positions selected from the group consisting of positions 33, 41, 68, 74, 94, 201, 225, 424, 445, 448 and 453, an amino acid that does not correspond to the amino acid of the corresponding position(s) of the sequence according to SEQ ID NO. 2.
Sentence 4. The enzyme according to any one of the preceding sentences, wherein the amino acid sequence at position 33 has an arginine or a tryptophan or a histidine or a phenylalanine, and/or at position 41 has a tryptophan, and/or at position 68 has an asparagine, and/or at position 74 has a tryptophan, and/or at position 94 has an isoleucine, and/or at position 175 has an alanine, and/or at position 201 has a phenylalanine, and/or at position 217 has an arginine, and/or at position 221 has a glycine, and/or at position 225 has a threonine and/or at position 229 has a cysteine, and/or at position 317 has an isoleucine, and/or at position 328 has an arginine, and/or at position 424 has a valine, and/or at position 445 has an alanine, and/or at position 448 has a histidine and/or at position 453 has an aspartate or a cysteine or an asparagine or a glutamine or a glutamate or a lysine or an arginine or a serine or an asparagine, and/or at position 454 has an asparagine, and/or at position 457 has a glycine, and/or at position 507 has a proline.
Sentence 5. The enzyme according to any one of the preceding sentences, wherein the amino acid sequence at position 33 has an arginine or a tryptophan, and/or at position 68 has an asparagine, and/or at position 74 has a tryptophan, and/or at position 201 has a phenylalanine, and/or at position 225 has a threonine, and/or at position 424 has a valine, and/or at position 445 has an alanine, and/or at position 448 has a histidine, and/or at position 453 has an aspartate or a cysteine.
Sentence 6. An enzyme for reducing the amount of acrylamide in a preparation comprising or consisting of an amino acid consensus sequence according to SEQ ID NO. 1, wherein said amino acid sequence is not a sequence according to SEQ ID NO. 2, and wherein said enzyme comprises or consists of an amino acid sequence having a sequence identity of at least 95%, 96%, 97%, 98% or 99% to a sequence selected from the group consisting of the sequences according to SEQ ID NO. 3 to SEQ ID NO. 41.
Sentence 7. The enzyme of sentence 6, wherein the enzyme comprises or consists of an amino acid sequence having a sequence identity of at least 95%, 96%, 97%, 98%, or 99% to a sequence selected from the group consisting of the sequences SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, and SEQ ID NO. 22.
Sentence 8. The enzyme of sentence 6 or 7, wherein the enzyme comprises or consists of an amino acid sequence having a sequence identity of at least 95%, 96%, 97%, 98%, or 99% to a sequence selected from the group consisting of the sequences SEQ ID NO. 19, SEQ ID NO. 20, SEQ ID NO. 21, and SEQ ID NO. 22.
Sentence 9. The enzyme according to any one of the preceding sentences, wherein the enzyme is an amidase.
Sentence 10. The enzyme according to any one of the preceding sentences, wherein said enzyme exhibits catalytic activity up to a temperature of 50° C. or more, preferably at least up to a temperature of 80° C., more preferably at least up to a temperature of 70° C., more preferably at least up to a temperature of 80° C.; and/or wherein said enzyme exhibits catalytic activity in a range of pH 4 to pH 7, preferably wherein said enzyme exhibits catalytic activity in a range of pH 4 to pH 6.5, more preferably in a range of pH 4.5 to pH 5.5; preferably wherein the enzyme has catalytic activity up to a temperature of 50° C. or more, preferably at least up to a temperature of 60° C., more preferably at least up to a temperature of 70° C., more preferably at least up to a temperature of 80° C., in a range of pH 4 to pH 7, preferably in a range of pH 4 to pH 6.5, more preferably in the range of pH 4.5 to pH 5.5.
Sentence 11. The enzyme according to any one of the preceding sentences comprising or consisting of an amino acid sequence having a sequence identity of at least 90%, 91%, 92%, 93%, 94%, 95%, 96%, 97%, 98%, 99% or 100% to a sequence selected from the group consisting of the sequences according to SEQ ID NO. 3 to SEQ ID NO. 41, preferably from the sequences according to SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21 and SEQ ID NO. 22, particularly preferably from the sequences according to SEQ ID NO. 19, SEQ ID NO. 20, SEQ ID NO. 21 and SEQ ID NO. 22.
Sentence 12. The enzyme according to any one of the preceding sentences, wherein the enzyme is an altered amidase from Pseudonocardia thermophila.
Sentence 13. A method for degrading acrylamide, preferably for reducing the amount of acrylamide in a preparation, consisting of or comprising the steps of: (i) providing an enzyme according to any one of sentences 1 to 12; (ii) providing a mixture, preferably a preparation, containing acrylamide and adding the enzyme of step (i); (iii) incubating the mixture resulting from step (ii) for at least 20 minutes, preferably at a temperature in the range of 40° C. to 80° C., more preferably at a temperature in the range of 45° C. to 75° C.; (iv) optionally: heating the incubated mixture resulting from step (iii) so that inactivation of the enzyme occurs, preferably by heating to a temperature of at least 90° C. and maintaining a temperature above 90° C. for at least 15 minutes, and optionally cooling the mixture; to obtain a product having a lower acrylamide content than the mixture/preparation provided in step (ii).
Sentence 14. A method according to sentence 13, wherein in step (ii) a preparation containing acrylamide is provided, wherein the acrylamide contained is preferably the product of a Maillard reaction, and wherein the preparation is a preparation serving pleasure or nourishment or a cosmetic preparation, or a semi-finished product for the production of such preparations, preferably wherein the preparation is selected from the group consisting of fried or deep-fried potato products, roasted cereals or products containing them, com products, coffee products, e.g. solid or liquid coffee extracts and green coffee, chicory extracts, cereal coffee products, coffee substitute products, snacks, wheat products, cosmetics, baked goods or pastries, e.g. biscuits, cookies, rusks, cereal bars, scones, ice cream cones, waffles, crumpets, gingerbread, crispbread and bread substitutes, pasta, rice, fish products, meat products, cereals, beer, nuts, complementary foods for children and infants, hair styling products, personal care products, hair care products, and facial care products.
Sentence 15. The method according to one of sentences 13 or 14, wherein the acrylamide content in the product obtained is < 2000 µg/kg, preferably < 850 µg/kg, particularly preferably < 500 µg/kg, in each case based on the total weight of the product.
Sentence 16. A method for producing a preparation serving pleasure or nourishment or a cosmetic preparation with reduced acrylamide content, preferably wherein the preparation is selected from the group consisting of fried or deep-fried potato products, roasted cereals or products containing them, corn products, coffee products, e.g., solid or liquid coffee extracts and green coffee, chicory extracts, cereal coffee products, coffee substitute products, snacks, wheat products, cosmetics, baked goods and pastries, e.g. biscuits, cookies, rusks, cereal bars, scones, ice cream cones, waffles, crumpets, gingerbread, crispbread and bread substitutes, pasta, rice, fish products, meat products, cereals, beer, nuts, complementary foods for children and infants, hair styling products, personal care products, hair care products, and facial care products consisting of or including the steps of: (I) (a) providing a product obtained by a method according to any one of sentences 13 to 15; and (b) further processing the product and/or adding one or more further ingredients to obtain the preparation serving pleasure or nourishment, or the cosmetic preparation; or (II) (i) providing an enzyme according to any one of sentences 1 to 9; (ii) providing a preparation serving pleasure or nourishment, or a cosmetic preparation, containing acrylamide and adding the enzyme of step (i); (iii) incubating the preparation resulting from step (ii) for at least 20 minutes, preferably at a temperature ranging from 40° C. to 80° C., more preferably at a temperature ranging from 45° C. to 75° C.; (iv) optionally: heating the incubated preparation resulting from step (iii) so that inactivation of the enzyme occurs, preferably by heating to a temperature of at least 90° C. and maintaining a temperature above 90° C. for at least 15 minutes, and optionally cooling the preparation; to obtain a preparation having a lower acrylamide content than the preparation provided in step (ii).
Sentence 17: Use of an enzyme according to any one of sentences 1 to 12 for the degradation of acrylamide and/or for the production of a preparation serving pleasure or nourishment, or a cosmetic preparation with a reduced acrylamide content, preferably < 2000 µg/kg, preferably < 850 µg/kg, particularly preferably < 500 µg/kg, in each case based on the total weight of the preparation.
Sentence 18: A preparation serving pleasure or nourishment, or a cosmetic preparation, produced or producable by a method according to any one of sentences 13 to 16, wherein the acrylamide content is < 2000 µg/kg, preferably < 850 µg/kg, particularly preferably < 500 µg/kg, in each case based on the total weight of the preparation.

The present invention is explained in more detail below with the help of selected non-limiting examples.

### 1. DEVELOPMENT OF AN ENZYME FOR THE DEGRADATION OF ACRYLAMIDE IN COFFEE

### 1.1 Manufacture of Enzyme Preparations

The genes encoding an amidase and its variants are cloned into the expression plasmid pLE1A17 (Novagen). Cells of E. coli BL21 (DE3) are then transformed with these plasmids.

Cells are cultured in ZYM505 medium (F. William Studier, Protein Expression and Purification 41 (2005) 207-234)) with the addition of kanamycin (50 mg/l) at 37° C. and enzyme expression is induced upon reaching the logarithmic growth phase with IPTG to 0.1 mM final concentration. Cells are further cultured at 30° C. for approximately 20 h after induction.

Cells are harvested by centrifugation and digested in lysis buffer (50 mM potassium phosphate, pH 7.2; 2 mM MgCl₂; 0.5 mg/mL lysozyme; 0.02 U/µL nucleanase). Digestion was performed mechanically either by multiple freezing and thawing in liquid nitrogen or by ultrasound. After centrifugation and separation of the insoluble components, the soluble enzyme-containing crude extract was obtained.

### 1.2 Determination of Enzyme Activity

For the standard determination of amidase activity, the release of ammonia from acrylamide at pH 5.5 and 40° C. is followed. One amidase unit corresponds to the release of 1 µmol ammonia per minute from 25 mM acrylamide in 50 mM sodium acetate buffer pH 5.5 at 40° C. Quantification of the ammonia released was performed using, for example, the Rapid Ammonia Kit from Megazymes. The activities given in U/mL refer to mL of crude extract with an optical density (measured at 600 nm) of 100.

The activity is determined analogously at other pH values (pH 5.0) and temperatures (50/60° C.). The parameters that have changed compared to the standard activity are indicated separately.

### 1.3.1 Determination of Enzyme Stability

### 1.3.1 Determination of Tₘ values at Different pH Values

To record the temperature stability of an amidase, the Tₘ 50 value is determined. For this purpose, a crude extract containing enzyme is incubated in 50 mM sodium acetate buffer (pH 4.5 to pH 5.5) for 15 minutes at different temperatures ranging from 25° C. to 85° C. The crude extracts are then incubated on ice for 15 minutes, centrifuged, and the supernatant is then used to perform the activity determination under standard conditions as described in section 1.2. The activity value of the untreated sample is set to 100% and all other values are normalized to this. The Tₘ 50 value corresponds to the temperature after which the enzyme is still 50% active.

### 1.3.2 Determination of Stability at Different pH Values and Temperatures

For the studies on the long-term stability of the enzymes, the crude extracts are incubated at a specific pH (e.g., in the range of pH 4.5 to 5.5) and a specific temperature (e.g., in the range of 50 to 75° C.) for an extended period of time. Regular sampling is performed over a 24 h period, during which samples are mixed with 1 volume equivalent of 100 mM NaAc buffer pH 5.5 and immediately frozen in liquid nitrogen. After thawing, the samples are centrifuged and the supernatant is used to perform activity determinations under standard conditions as described in section 1.2. The percent residual activity is calculated by comparing the activity values determined after incubation with the activity of the untreated sample at time 0 h, where the activity value of the untreated sample is set to 100% and the activity values determined after incubation are given as percent residual activity in relation to it.

### 1.4 Examination of the Enzyme Variants

Based on the wild-type sequence with SEQ ID NO. 2, single mutants were generated and a selection of amino acid substitutions were combined from these.

TABLE 1

Results of the stability and activity determinations (standard determination)

| SEQ ID NO. | Stability | | Stability | | Activity | |
|---|---|---|---|---|---|---|
| | Tm50 [°C] | Tm50 [°C] | Residual activity [%] after 24 h | | pH 5.0, 40° C. | pH 5.5, 40° C. |
| | pH 5.0 | pH 5.5 | pH 5.0, 50° C. pH 5.5, 60° C. | | U/mL | U/mL |
| 2 | 33 | 51 | 0 | 0 | 41 | 69 |
| 3 | n.d. | 55 | n.d. | n.d. | 50 | 87 |
| 4 | n.d. | 57 | n.d. | n.d. | 45 | 87 |
| 5 | n.d. | 58 | n.d. | n.d. | 45 | 85 |
| | | | n.d. | n.d. | n. | |
| 6 | n.d. | 57 | | | d. | n.b |
| 7 | n.d. | 54 | n.d. | n.d. | 51 | 100 |
| 8 | n.d. | 52 | n.d. | n.d. | 80 | 136 |
| 9 | 61 | 72 | 61 | 0 | 85 | 121 |
| 10 | 64 | 73 | 68 | 36 | 67 | 89 |
| 11 | 64 | 72 | 82 | 45 | 51 | 79 |
| 12 | 56 | 69 | 37 | 0 | 69 | 108 |
| 13 | 64 | 74 | 57 | 0 | 53 | 79 |

If no data are given in the tables or the term n.d. (for “not determined”) is used, these were not recorded for the corresponding enzyme variant.

It can be seen that stability at temperatures above 50° C., as well as activity at high temperatures and an acidic pH could be achieved for the enzymes of the invention.

Further investigations revealed that the enzymes according to the invention, in particular the enzymes described herein as preferred, are also otherwise particularly suitable for the purposes and requirements described herein, in particular by way of introduction.

### 1.5 Investigation of Different Amino Acid Substitutions and Their Influence on the Stability and Activity of the Enzyme

The enzyme with SEQ ID NO. 11 was used as a template and single mutants were generated based on it. The individual amino acid substitutions show different effects on activity and stability of the enzymes. For this characterization, the mutants were selected based on the two criteria of stability and activity. The respective properties strongly depend on the substituted amino acids and their positions. In particular, these studies resulted in the enzymes particularly suitable according to the invention (as described herein).

TABLE 2

Results of the amino acid substitutions compared to the enzyme with SEQ ID NO. 11 (according to the invention, but not particularly preferred). The HIT criterion describes in each case the property on which selection was made. This is, on the one hand, an increased stability compared to an enzyme with SEQ ID NO. 11, on the other hand, an increased activity compared to an enzyme with SEQ ID NO. 11

| Substitution to SEQ ID NO. 11 | SEQ ID NO. | Hit criterion | [%] residual stability to SEQ ID NO. 11 | [%] residual stability to SEQ ID NO. 11 |
|---|---|---|---|---|
| P454N | 23 | Stability | 226 | 58 |
| V457G | 24 | Stability | 216 | 49 |
| L424V | 25 | Stability | 213 | 65 |
| A453D | 26 | Stability | 198 | 77 |
| S33Y | 27 | Stability | 172 | 77 |
| G175A | 28 | Stability | 154 | 75 |
| A507P | 29 | Stability | 153 | 83 |
| A453S | 30 | Stability | 147 | 103 |
| V94I | 31 | Stability | 147 | 109 |
| V317I | 32 | Stability | 142 | 78 |
| Y201F | 33 | Stability | 135 | 100 |
| - | 11 | Template | 100 | 100 |
| M448H | 34 | Activity | 51 | 186 |
| A453R | 35 | Activity | 0 | 161 |
| P221G | 36 | Activity | 0 | 150 |
| T217R | 37 | Activity | 0 | 135 |
| D328R | 38 | Activity | 15 | 133 |
| G445S | 39 | Activity | 36 | 132 |
| L229C | 40 | Activity | 0 | 130 |
| W41Y | 41 | Activity | 68 | 130 |

### 1.6 Investigation of Different Combinations of Mutations and Their Effect on the Activity and Astability of the Enzyme Nvariants

Several single mutants were generated starting from SEQ ID NO. 11, and a selection of amino acid substitutions was combined in recombination banks. A selection of suitable variants was then screened for stability and activity.

TABLE 3

Results of the different combination of amino acid substitutions and their effect on the stability and activity of enzymes according to the invention

| SEQ ID NO. | Stabilität | | | Stabilität | | Aktiviät |
|---|---|---|---|---|---|---|
| | Tm50 [°C] | Tm50 [°C] | Tm50 [°C] | Residual activity [%] after 18 h | | pH 5.5, 40° C. |
| | pH 4.5 | pH 5.0 | pH 55 | pH 45, 60° C. | pH 5.0, 70° C. | U/mL |
| 11 | 52 | 64 | 72 | 0 | 2 | 79 |
| 14 | 65 | 76 | 82 | 21 | 36 | 118 |
| 15 | 67 | 77 | 84 | 19 | 44 | 88 |
| 16 | 70 | 80 | 85 | 40 | 58 | 95 |
| 17 | 57 | 67 | 77 | 2 | 7 | 170 |
| 18 | 55 | 70 | 79 | 10 | 23 | 184 |

### 1.7 Saturation Mutagenesis at Position 453

All amino acid substitutions at position 453 in the variant with SEQ ID NO. 16 were examined. Variants with increased activity were obtained, which are the result of improved soluble expression in E. coli. The stability of the enzymes was maintained.

TABLE 4

Results of saturation mutagenesis and their effect on the stability and activity of enzymes according to the invention

| SEQ ID NO. | Stability | | Stability | | Activity |
|---|---|---|---|---|---|
| | Tm50 [°C] | Tm50 [°C] | Residual activity [%] after 22 h | | pH 5.5, 40° C. |
| | pH 4.5 | pH 5.0 | pH 4.5, 60° C. | pH 5.0, 70° C. | U/mL |
| 16 | 70 | 80 | 22 | 53 | 95 |
| 19 | 68 | 80 | 35 | 62 | 191 |
| 20 | 67 | 79 | 28 | 65 | 168 |
| 21 | 67 | 79 | 34 | 67 | 102 |
| 22 | 69 | 80 | 41 | 60 | 161 |

### 2. PRODUCTION OF EXEMPLARY PRODUCTS ACCORDING TO THE INVENTION

### 2.1 Process for the Production of an Acrylamide-Reduced Coffee Product

### 2.1.1 Production of a Coffee Extract

Coffee beans of the Brazil Arabica coffee variety are roasted to a color value of 110 according to Neuhaus Neotec Colortest II. By roasting the coffee beans at temperatures between 145° C. and 230° C. acrylamide is formed. The roasted coffee is ground on the VT6 coffee grinder (Mahlkönig) at level 13. The ground coffee is first poured into the percolator of an extraction unit and then filled with 70 L of water, which has a temperature of 85° C. The mixture is left to swell for one hour. Then the extraction is started at 85° C. 100 L of water is passed through the percolator at 85° C. and 6 bar overpressure into a collecting vessel. The hot water dissolves out the soluble components of the coffee, including acrylamide.

### 2.1.2 Enzymatic Treatment of the Coffee Extract

Following the extraction, the coffee extract obtained is heated to a temperature of 70° C. in the collecting vessel and kept at this temperature. An enzyme according to the invention, e.g. an enzyme according to SEQ ID NO. 22, is then added. The enzyme is added in an amount such that an enzyme concentration of 1000 U/L is employed.

The enzyme is added to the extract, stirred and incubated at 70° C. for 30 minutes. After incubation, the extract is heated to 95° C. for 15 minutes to inactivate the amidase. The extract is then cooled down and prepared for freeze-drying.

Depending on the pH of the coffee extract, different reduction rates in relation to the amount of acrylamide are achieved. Depending on the amount of enzyme used, acrylamide concentrations below the detection limit can also be achieved. However, it was the object of the present invention to provide an enzyme which is also suitable for use in a coffee matrix from an economic point of view. For example, at a pH of 4.8 and 1000 U/L of the enzyme used, an acrylamide reduction of 60% was achieved. At a pH of 5.3 and 1000 U/L of the enzyme used, an acrylamide reduction of 90% was achieved. Thus, acrylamide-reduced products with only 554 and 129 µg/kg acrylamide content, respectively, were obtained.

### 2.1.3 Further Processing of the Coffee Extract After Enzyme Treatment

After extraction and enzyme treatment, the coffee extract is concentrated using freeze concentration or evaporation. Concentration is an intermediate step to increase the solids content in the extract, since the extract obtained after extraction has a solids content of about 2 - 6 wt.%. For fluid bed drying, a solids content of at least 20 wt% is required. For freeze drying, a higher solids content is advantageous, but not absolutely necessary.

The concentrated extract is then dried by freeze-drying or fluidized-bed drying to obtain the acrylamide-reduced end product (dried dissolved coffee), which usually has a solids content of about 96% by weight.

### 2.2 Process for the Preparation of an Acrylamide-Reduced Coffee Substitute Product

As a coffee substitute product can be used, for example, the chicory. The root of the chicory plant is used for this purpose. This is dried, crushed and roasted like coffee at temperatures between 150 and 200° C. and then ground. Further processing to obtain the soluble, acrylamide-reduced extract is carried out in the same way as described for coffee in Example 2.1.2.

The dried, soluble chicory extract can be used as a final product or as a blended additive, for example for cereal coffee or coffee mix products.

### 3. DETERMINATION OF ACRYLAMIDE CONTENTS AS WELL AS ACRYLAMIDE REDUCTION

Acrylamide reduction is determined by extracting a sample of a particular roasted coffee with hot water. The extract is then divided into two portions and only one portion is treated with amidase and incubated. The second portion is treated the same, except for the addition of amidase, and serves as a reference sample. At the end of the incubation period, the reaction is stopped by heating once to a temperature that safely denatures the enzyme. The acrylamide analysis in both extracts is carried out according to DIN EN ISO 18862 with LC-MS/MS. The reduction rate is calculated from the acrylamide contents in the treated sample and in the reference sample.

### 4. SENSORY EVALUATION

Comparative sensory testing of different coffee extracts and extracts of coffee substitutes by a trained sensory panel revealed no perceptible sensory differences between the untreated and treated extracts.

### 5. INVESTIGATION OF DIFFERENT AMINO ACID SUBSTITUTIONS AND THEIR INFLUENCE ON THE STABILITY AND ACTIVITY OF SEQ ID NO.2:

The enzyme preparations were prepared analogously to experiment 1 (cf. 1.1). The same conditions were used for the determination of enzyme activity (cf. 1.2) and enzyme stability (cf. 1.3).

Based on the wildtype sequence having SEQ ID NO. 2, the influence of different amino acid substitutions and their impact on activity (Table 4) and stability (Table 5) was determined in comparison to SEQ ID NO.2.

TABLE 5

Effect of different amino acid substitutions on activity of SEQ ID NO:2

| SEQ ID NO. | [%] rel. activity to SEQ ID NO. 2 | |
|---|---|---|
| | pH 5.0, 40° C. | pH 5.5, 40° C. |
| 2 | 100 | 100 |
| 42 | 132 | 122 |
| 43 | 142 | n.b. |
| 46 | 151 | n.b. |
| 47 | 122 | n.b. |
| 48 | 146 | 110 |
| 49 | 133 | 105 |
| 50 | 171 | 111 |
| 51 | 129 | 112 |
| 52 | 111 | n.b. |
| 54 | 174 | 124 |
| 55 | 139 | 104 |

TABLE 6

Effect of different amino acid substitutions on the stability of SEQ ID NO.2

| SEQ ID NO. | Tm50 [°C] | |
|---|---|---|
| | pH 5.0 | pH 5.5 |
| 2 | 33 | 51 |
| 42 | 51 | 61 |
| 43 | 48 | 57.5 |
| 44 | 43.5 | 55.5 |
| 45 | 41.5 | 55 |
| 46 | 48 | 54.5 |
| 47 | 46 | 54.5 |
| 48 | 45.5 | 54 |
| 49 | 45 | 54 |
| 50 | 45 | 53.5 |
| 51 | 47.5 | 53 |
| 52 | 45 | 53 |
| 53 | 44 | 53 |
| 54 | 43 | 52.5 |
| 55 | 44.5 | 52.5 |

1. An enzyme for reducing the amount of acrylamide in a preparation, comprising an amino acid sequence having a sequence identity of at least 86% to SEQ ID NO. 2, wherein the amino acid sequence has, compared to SEQ ID NO. 2, at least one amino acid substitution at a position located in one of the following sequence segments of SEQ ID NO. 2:
(a) Position L424 to position A507;
(b) position S33 to position A74; or
(c) Position G175 to position L229.

2. The enzyme of claim 1, which, compared to an enzyme having SEQ ID NO. 2,
(i) exhibits higher enzyme activity in the catalyzed release of ammonia from 25 mM acrylamide in 50 mM sodium acetate buffer pH 5.5 at 40° C.;
(ii) exhibits higher enzyme activity in the catalyzed release of ammonia from 25 mM acrylamide in 50 mM sodium acetate buffer pH 5 at 40° C.;
(iii) has a higher Tₘ 50 value in 50 mM sodium acetate buffer pH 5.5;
(iv) has a higher Tₘ 50 value in 50 mM sodium acetate buffer pH 5; and/or
(v) has higher residual activity after incubation for 24 h at 50° C. in 50 mM sodium acetate buffer pH 5.0.

3. The enzyme of claim 1, wherein the amino acid sequence has, compared to SEQ ID NO. 2, at least two amino acid substitutions of which
the first amino acid substitution is located in a position in sequence section (a) and the second amino acid substitution is also located in a position in sequence section (a);
the first amino acid substitution is located in a position in sequence section (a) and the second amino acid substitution is located in a position in sequence section (b);
the first amino acid substitution is located in a position in sequence section (a) and the second amino acid substitution is located in a position in sequence section (c);
the first amino acid substitution is located in a position in sequence section (b) and the second amino acid substitution is also located in a position in sequence section (b);
the first amino acid substitution is located in a position in sequence section (b) and the second amino acid substitution is located in a position in sequence section (c); or
the first amino acid substitution is located in a position in sequence section (c) and the second amino acid substitution is also located in a position in sequence section (c).

4. The enzyme of claim 1, wherein the amino acid sequence has, compared to SEQ ID NO. 2, at least one amino acid substitution in a position located in one of the following sequence segments of SEQ ID NO. 2:
(a-1) Position G445 to V465;
(a-2) Position L424 to R444;
(a-3) Position P487 to A507; er
(a-4) Position P466 to A486;
(b-1) Position S33 to position P53;
(b-2) Position T54 to position A74.
(c-1) Position G212 to position L229;
(c-2) Position G193 to position H211; or
(c-3) Position G175 to position G192.

5. (canceled)

6. (canceled)

7. The enzyme of claim 4, wherein the amino acid sequence has, compared to SEQ ID NO. 2, at least two amino acid substitutions of which
the first amino acid substitution is located in a position in sequence section (a-1) and the second amino acid substitution is also located in a position in sequence section (a-1);
the first amino acid substitution is located in a position in sequence section (a-1) and the second amino acid substitution is located in a position in sequence section (a-2);
the first amino acid substitution is located in a position in sequence section (a-1) and the second amino acid substitution is located in a position in sequence section (a-3);
the first amino acid substitution is located in a position in sequence section (a-1) and the second amino acid substitution is located in a position in sequence section (b-1);
the first amino acid substitution is located in a position in sequence section (a-1) and the second amino acid substitution is located in a position in sequence section (b-2);
the first amino acid substitution is located in a position in sequence section (a-1) and the second amino acid substitution is located in a position in sequence section (c-1);
the first amino acid substitution is located in a position in sequence section (a-1) and the second amino acid substitution is located in a position in sequence section (c-2);
the first amino acid substitution is located in a position in sequence section (a-2) and the second amino acid substitution is located in a position in sequence section (a-3);
the first amino acid substitution is located in a position in sequence section (a-2) and the second amino acid substitution is located in a position in sequence section (b-1);
the first amino acid substitution is located in a position in sequence section (a-2) and the second amino acid substitution is located in a position in sequence section (b-2);
the first amino acid substitution is located in a position in sequence section (a-2) and the second amino acid substitution is located in a position in sequence section (c-1);
the first amino acid substitution is located in a position in sequence section (a-2) and the second amino acid substitution is located in a position in sequence section (c-2);
the first amino acid substitution is located in a position in sequence section (a-3) and the second amino acid substitution is located in a position in sequence section (b-1);
the first amino acid substitution is located in a position in sequence section (a-3) and the second amino acid substitution is located in a position in sequence section (b-2);
the first amino acid substitution is located in a position in sequence section (a-3) and the second amino acid substitution is located in a position in sequence section (c-1);
the first amino acid substitution is located in a position in sequence section (a-3) and the second amino acid substitution is located in a position in sequence section (c-2);
the first amino acid substitution is located in a position in sequence section (b-1) and the second amino acid substitution is also located in a position in sequence section (b-1);
the first amino acid substitution is located in a position in sequence section (b-1) and the second amino acid substitution is located in a position in sequence section (b-2);
the first amino acid substitution is located in a position in sequence section (b-1) and the second amino acid substitution is located in a position in sequence section (c-1);
the first amino acid substitution is located in a position in sequence section (b-1) and the second amino acid substitution is located in a position in sequence section (c-2);
the first amino acid substitution is located in a position in sequence section (b-2) and the second amino acid substitution is also located in a position in sequence section (b-2);
the first amino acid substitution is located in a position in sequence section (b-2) and the second amino acid substitution is located in a position in sequence section (c-1);
the first amino acid substitution is located in a position in sequence section (b-2) and the second amino acid substitution is located in a position in sequence section (c-2); or
the first amino acid substitution is located in a position in sequence section (c-1) and the second amino acid substitution is located in a position in sequence section (c-2).

8. The enzyme of claim 1, wherein the amino acid sequence has, compared to SEQ ID NO. 2, at least three amino acid substitutions of which
the first amino acid substitution is located in a position in sequence section (a), the second amino acid substitution is also located in a position in sequence section (a), and the third amino acid substitution is also located in a position in sequence section (a);
the first amino acid substitution is located in a position in sequence section (a), the second amino acid substitution is also located in a position in sequence section (a), and the third amino acid substitution is located in a position in sequence section (b);
the first amino acid substitution is located in a position in sequence section (a), the second amino acid substitution is also located in a position in sequence section (a), and the third amino acid substitution is located in a position in sequence section (c);
the first amino acid substitution is located in a position in sequence section (a), the second amino acid substitution is located in a position in sequence section (b), and the third amino acid substitution is also located in a position in sequence section (b);
the first amino acid substitution is located in a position in sequence section (a), the second amino acid substitution is located in a position in sequence section (b), and the third amino acid substitution is located in a position in sequence section (c);
the first amino acid substitution is located in a position in sequence section (a), the second amino acid substitution is located in a position in sequence section (c), and the third amino acid substitution is also located in a position in sequence section (c);
the first amino acid substitution is located in a position in sequence section (b), the second amino acid substitution is also located in a position in sequence section (b), and the third amino acid substitution is also located in a position in sequence section (b);
the first amino acid substitution is located in a position in sequence section (b), the second amino acid substitution is also located in a position in sequence section (b), and the third amino acid substitution is located in a position in sequence section (c); or
the first amino acid substitution is located in a position in sequence section (c), the second amino acid substitution is also located in a position in sequence section (c), and the third amino acid substitution is also located in a position in sequence section (c).

9. The enzyme of claim 1, wherein the amino acid sequence has, compared to SEQ ID NO. 2, no amino acid substitution at least in one of the following positions: E24, S25, D26, L27, P28, A32, S33, T35, L 37, L38, S40, W41, N42, K43, V44, E45, E46, Y48, A49, E50, V51, A52, P53, T54, Q57, S59, W60, T61, R62, P63, A65, E66, D67, D68, K69, A72, W73, V75, Q76, T77, S78, 179, T80, E81, T82, S83, E84, G85, P86, L87, A88, T91, V92, A93, V94, K95, P156, S170, S194, I330, N348, and E509.

10. (canceled)

11. The enzyme of claim 1, wherein the amino acid sequence has, compared to SEQ ID NO. 2, an amino acid substitution in position G445; A453; L424; M448; A507; S33; W41; D68; A74; V94; Y201; P221; S225; G175; T217; L229; V317; D328 ; P454; or V457.

12-36. (canceled)

37. The enzyme of claim 1, wherein the amino acid sequence has, compared to SEQ ID NO. 2, at least two amino acid substitutions selected from positions S33, D68, A74, S225 and G445.

38-51. (canceled)

52. The enzyme of claim 1, wherein the amino acid sequence has, compared to SEQ ID NO. 2, at least two amino acid substitutions in positions
S33 and W41; S33 and D68; S33 and A74; S33 and V94; S33 and Y201; S33 and S225; S33 and L424; S33 and G445; S33 and M448; S33 and A453; or S33 and A507;
W41 and D68; W41 and A74; W41 and V94; W41 and Y201; W41 and S225; W41 and L424; W41 and G445; W41 and M448; W41 and A453; or W41 and A507;
D68 and A74; D68 and V94; D68 and Y201; D68 and S225; D68 and L424; D68 and G445; D68 and M448; D68 and A453; or D68 and A507;
A74 and V94; A74 and Y201; A74 and S225; A74 and L424; A74 and G445; A74 and M448; A74 and A453; or A74 and A507;
V94 and Y201; V94 and S225; V94 and L424; V94 and G445; V94 and M448; V94 and A453; or V94 and A507;
Y201 and S225; Y201 and L424; Y201 and G445; Y201 and M448; or Y201 and A453;
S225 and L424; S225 and G445; S225 and M448; S225 and A453; or S225 and A507;
L424 and G445; L424 and M448; L424 and A453; or L424 and A507;
G445 and M448; G445 and A453; or G445 and A507;
M448 and A453; or M448 and A507; or
A453 and A507.

53. The enzyme of claim 1, wherein the amino acid sequence has, compared to SEQ ID NO. 2, at least one of the amino acid substitutions selected from positions S33, D68, A74, G175, S225, L424, G445, A453, and A507.

54. The enzyme of claim 1, wherein the amino acid sequence has a sequence identity to SEQ ID NO. 2 of at least 90%.

55. The enzyme of claim 1, wherein the amino acid sequence has a sequence identity of at least 95%, to SEQ ID NO. 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46. 47, 48, 49, 50, 51, 52, 53, 54 or 55.

56. A method for degrading acrylamide, the steps:
(i) Providing an enzyme of claim 1;
(ii) providing a mixture, containing acrylamide and adding the enzyme of step (i);
(iii) incubating the mixture resulting from step (ii) for at least 20 minutes, in the range of 40° C. to 80° C.;
(iv) optionally, heating the incubated mixture resulting from step (iii) so that inactivation of the enzyme occurs, and optionally cooling the mixture;
to obtain a product having a lower acrylamide content than the mixture or preparation provided in step (ii).

57. The method of claim 56, wherein in step (ii) a mixture containing acrylamide is provided, wherein the acrylamide contained is the product of a Maillard reaction.

58. The method of claim 56, wherein the acrylamide content in the product obtained is < 2000 µg/kg, based on the total weight of the product.

59. A method for preparing a preparation serving pleasure or nourishment, or a cosmetic preparation, with reduced acrylamide content, preferably wherein the preparation is selected from the group consisting of fried or deep-fried potato products, roasted cereals or products containing them, corn products, coffee products, chicory extracts, cereal coffee products, coffee substitute products, snacks, wheat products, cosmetics, baked goods or pastries, pasta, rice, fish products, meat products, cereals, beer, nuts, complementary foods for children and infants, hair styling products, personal care products, hair care products, and facial care products, comprising the steps:
(a) providing a product obtained by a process of claim 56; and
(b) further processing of the product and/or adding one or more additional ingredients to obtain the preparation serving pleasure or nourishment, or the cosmetic preparation.

60. (canceled)

61. (canceled)

62. The method of claim 56, wherein in step (ii) a mixture containing acrylamide is provided, wherein the mixture is a preparation serving pleasure or nourishment, or a cosmetic preparation, or a semi-finished product for the production of such preparations, preferably wherein the preparation is selected from the group consisting of fried or deep-fried potato products, roasted cereals or products containing them, corn products, coffee products, chicory extracts, cereal coffee products, coffee substitute products, snacks, wheat products, cosmetics, baked goods or pastries, pasta, rice, fish products, meat products, cereals, beer, nuts, complementary foods for children and infants, hair styling products, personal care products, hair care products, and facial care products.

63. A method for preparing a preparation serving pleasure or nourishment, or a cosmetic preparation, with reduced acrylamide content, preferably wherein the preparation is selected from the group consisting of fried or deep-fried potato products, roasted cereals or products containing them, corn products, coffee products, chicory extracts, cereal coffee products, coffee substitute products, snacks, wheat products, cosmetics, baked goods or pastries, pasta, rice, fish products, meat products, cereals, beer, nuts, complementary foods for children and infants, hair styling products, personal care products, hair care products, and facial care products, comprising the steps:
(i) providing an enzyme of claim 1;
(ii) providing a preparation serving pleasure or nourishment, or a cosmetic preparation, containing acrylamide and adding the enzyme of step (i);
(iii) incubating the preparation resulting from step (ii) for at least 20 minutes in the range of 40° C. to 80° C.;
(iv) optionally, heating the incubated preparation resulting from step (iii) so that inactivation of the enzyme occurs, and optionally cooling the preparation; to obtain a preparation having a lower acrylamide content than the preparation provided in step (ii).

64. The enzyme of claim 1, wherein the amino acid sequence has, compared to SEQ ID NO. 2, an amino acid substitution in position G445.


SEQUENCE LISTING (56 sequences)

SEQ ID NO: 1 - protein, 512 aa, Artificial Sequence
  Synthetic Construct
  misc_feature (33)..(33): Xaa can be any naturally occurring amino acid
  misc_feature (41)..(41): Xaa can be any naturally occurring amino acid
  misc_feature (68)..(68): Xaa can be any naturally occurring amino acid
  misc_feature (74)..(74): Xaa can be any naturally occurring amino acid
  misc_feature (94)..(94): Xaa can be any naturally occurring amino acid
  misc_feature (175)..(175): Xaa can be any naturally occurring amino acid
  misc_feature (201)..(201): Xaa can be any naturally occurring amino acid
  misc_feature (217)..(217): Xaa can be any naturally occurring amino acid
  misc_feature (221)..(221): Xaa can be any naturally occurring amino acid
  misc_feature (225)..(225): Xaa can be any naturally occurring amino acid
  misc_feature (229)..(229): Xaa can be any naturally occurring amino acid
  misc_feature (317)..(317): Xaa can be any naturally occurring amino acid
  misc_feature (328)..(328): Xaa can be any naturally occurring amino acid
  misc_feature (424)..(424): Xaa can be any naturally occurring amino acid
  misc_feature (445)..(445): Xaa can be any naturally occurring amino acid
  misc_feature (448)..(448): Xaa can be any naturally occurring amino acid
  misc_feature (453)..(454): Xaa can be any naturally occurring amino acid
  misc_feature (457)..(457): Xaa can be any naturally occurring amino acid
  misc_feature (507)..(507): Xaa can be any naturally occurring amino acid
MSIHMPDPDAVARMAAEGRFGIPESDLPTYHAXVTGLLGSXNKVEELYAEVAPTPPQRSWTRPDAEDXKLGAWXVQTSITETSEGPLAGRTVAXKDNVAVAGVPMMNGSRTLEGFVPAEDATVVSRLLAAGATIAGKSVCEDLCFSGGSHTSKPGPVHNPWDMSRSAGGSSSGSXALVAAGEVDMAIGGDQGGSIRIPSAXCGTVGHKPTHGLVPYXGGFXIEQXIDHXGPITRTVADAALMLSVIAGRDGLDPRQPDVVEVQDYVGALAESVSGLRIGVLQEGFGHPNSEPEVDDTVRAAVGTLREAGFTVEDVSXPWHLHATAIWXVIATEGGLWQMVEGNAYGMNWKGHYDPSLIAYYGRKWREDPAQFSETVKLVALAGRYALTTQYGRHYAMARNLAPKLVAAYDAALSNYDVLVMPTXPMRATVLPGPDAPVEEILARXLEXLANTXXFDXTGHPACSVPAGLADGLPVGLMIVGKHFDDATVLKVANAFEQAVGGFPTPXERSSA

SEQ ID NO: 2 - protein, 513 aa, Pseudonocardia thermophila
MSIHMPDPDAVARMAAEGRFGIPESDLPTYHASVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAERSSA

SEQ ID NO: 3 - protein, 513 aa, Artificial Sequence
  Synthetic Construct
MSIHMPDPDAVARMAAEGRFGIPESDLPTYHASVTGLLGSWNKVEELYAEVAPTPPQRSWTRPDAEDNKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAERSSA

SEQ ID NO: 4 - protein, 513 aa, Artificial Sequence
  Synthetic Construct
MSIHMPDPDAVARMAAEGRFGIPESDLPTYHASVTGLLGSWNKVEELYAEVAPTPPQRSWTRPDAEDDKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAERSSA

SEQ ID NO: 5 - protein, 513 aa, Artificial Sequence
  Synthetic Construct
MSIHMPDPDAVARMAAEGRFGIPESDLPTYHASVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 6 - protein, 513 aa, Artificial Sequence
  Synthetic Construct
MSIHMPDPDAVARMAAEGRFGIPESDLPTYHAFVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAERSSA

SEQ ID NO: 7 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAERSSA

SEQ ID NO: 8 - protein, 513 aa, Artificial Sequence
  Synthetic Construct
MSIHMPDPDAVARMAAEGRFGIPESDLPTYHASVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARSLEMLANTAPFDVTGHPACSVPAGLADGLPVGLMIVGKHFDDATVLKVANAFEQAVGGFPTPAAERSSA

SEQ ID NO: 9 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDDKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARSLEMLANTCPFDVTGHPACSVPAGLADGLPVGLMIVGKHFDDATVLKVANAFEQAVGGFPTPAAERSSA

SEQ ID NO: 10 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARSLEMLANTCPFDVTGHPACSVPAGLADGLPVGLMIVGKHFDDATVLKVANAFEQAVGGFPTPAAERSSA

SEQ ID NO: 11 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 12 - protein, 513 aa, Artificial Sequence
  Synthetic Construct
MSIHMPDPDAVARMAAEGRFGIPESDLPTYHASVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARSLEMLANTCPFDVTGHPACSVPAGLADGLPVGLMIVGKHFDDATVLKVANAFEQAVGGFPTPAAERSSA

SEQ ID NO: 13 - protein, 513 aa, Artificial Sequence
  Synthetic Construct
MSIHMPDPDAVARMAAEGRFGIPESDLPTYHAHVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAERSSA

SEQ ID NO: 14 - protein, 513 aa, Artificial Sequence
  Synthetic Construct
MSIHMPDPDAVARMAAEGRFGIPESDLPTYHARVTGLLGSYNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTDPFDVTGHPACSVPAGLADGLPVGLMIVGKHFDDATVLKVANAFEQAVGGFPTPPAERSSA

SEQ ID NO: 15 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTDPFDVTGHPACSVPAGLADGLPVGLMIVGKHFDDATVLKVANAFEQAVGGFPTPPAERSSA

SEQ ID NO: 16 - protein, 513 aa, Artificial Sequence
  Synthetic Construct
MSIHMPDPDAVARMAAEGRFGIPESDLPTYHAYVTGLLGSYNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTDPFDVTGHPACSVPAGLADGLPVGLMIVGKHFDDATVLKVANAFEQAVGGFPTPAAERSSA

SEQ ID NO: 17 - protein, 513 aa, Artificial Sequence
  Synthetic Construct
MSIHMPDPDAVARMAAEGRFGIPESDLPTYHARVTGLLGSYNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTAPFDVTGHPACSVPAGLADGLPVGLMIVGKHFDDATVLKVANAFEQAVGGFPTPAAERSSA

SEQ ID NO: 18 - protein, 513 aa, Artificial Sequence
  Synthetic Construct
MSIHMPDPDAVARMAAEGRFGIPESDLPTYHARVTGLLGSYNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTCPFDVTGHPACSVPAGLADGLPVGLMIVGKHFDDATVLKVANAFEQAVGGFPTPPAERSSA

SEQ ID NO: 19 - protein, 513 aa, Artificial Sequence
  Synthetic Construct
MSIHMPDPDAVARMAAEGRFGIPESDLPTYHAYVTGLLGSYNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTNPFDVTGHPACSVPAGLADGLPVGLMIVGKHFDDATVLKVANAFEQAVGGFPTPAAERSSA

SEQ ID NO: 20 - protein, 513 aa, Artificial Sequence
  Synthetic Construct
MSIHMPDPDAVARMAAEGRFGIPESDLPTYHAYVTGLLGSYNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTQPFDVTGHPACSVPAGLADGLPVGLMIVGKHFDDATVLKVANAFEQAVGGFPTPAAERSSA

SEQ ID NO: 21 - protein, 513 aa, Artificial Sequence
  Synthetic Construct
MSIHMPDPDAVARMAAEGRFGIPESDLPTYHAYVTGLLGSYNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTEPFDVTGHPACSVPAGLADGLPVGLMIVGKHFDDATVLKVANAFEQAVGGFPTPAAERSSA

SEQ ID NO: 22 - protein, 513 aa, Artificial Sequence
  Synthetic Construct
MSIHMPDPDAVARMAAEGRFGIPESDLPTYHAYVTGLLGSYNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTKPFDVTGHPACSVPAGLADGLPVGLMIVGKHFDDATVLKVANAFEQAVGGFPTPAAERSSA

SEQ ID NO: 23 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTANFDVTGHPACSVPAGLADGLPVGLMIVGKHFDDATVLKVANAFEQAVGGFPTPAAERSSA

SEQ ID NO: 24 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDGTGHPACSVPAGLADGLPVGLMIVGKHFDDATVLKVANAFEQAVGGFPTPAAERSSA

SEQ ID NO: 25 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 26 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTDPFDVTGHPACSVPAGLADGLPVGLMIVGKHFDDATVLKVANAFEQAVGGFPTPAAERSSA

SEQ ID NO: 27 - protein, 513 aa, Artificial Sequence
  Synthetic Construct
MSIHMPDPDAVARMAAEGRFGIPESDLPTYHAYVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 28 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSAALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 29 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPPAERSSA

SEQ ID NO: 30 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTSPFDVTGHPACSVPAGLADGLPVGLMIVGKHFDDATVLKVANAFEQAVGGFPTPAAERSSA

SEQ ID NO: 31 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 32 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSIPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 33 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 34 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEHLANTAPFDVTGHPACSVPAGLADGLPVGLMIVGKHFDDATVLKVANAFEQAVGGFPTPAAERSSA

SEQ ID NO: 35 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTRPFDVTGHPACSVPAGLADGLPVGLMIVGKHFDDATVLKVANAFEQAVGGFPTPAAERSSA

SEQ ID NO: 36 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFGIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 37 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYRGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 38 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWRVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 39 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARSLEMLANTAPFDVTGHPACSVPAGLADGLPVGLMIVGKHFDDATVLKVANAFEQAVGGFPTPAAERSSA

SEQ ID NO: 40 - protein, 513 aa, Artificial Sequence
  Synthetic Construct
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHCGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 41 - protein, 513 aa, Artificial Sequence
  Synthetic Construct
MSIHMPDPDAVARMAAEGRFGIPESDLPTYHARVTGLLGSYNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 42 - protein, 513 aa, Artificial Sequence
  Synthetic Construct
MSIHMPDPDAVARMAAEGRFGIPESDLPTYHASVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAERSSA

SEQ ID NO: 43 - protein, 513 aa, Artificial Sequence
  Synthetic Construct
MSIHMPDPDAVARMAAEGRFGIPESDLPTYHAYVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAERSSA

SEQ ID NO: 44 - protein, 513 aa, Artificial Sequence
  Synthetic Construct
MSIHMPDPDAVARMAAEGRFGIPESDLPTYHASVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSIPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAERSSA

SEQ ID NO: 45 - protein, 513 aa, Artificial Sequence
  Synthetic Construct
MSIHMPDPDAVARMAAEGRFGIPESDLPTYHASVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTEPFDVTGHPACSVPAGLADGLPVGLMIVGKHFDDATVLKVANAFEQAVGGFPTPAAERSSA

SEQ ID NO: 46 - protein, 513 aa, Artificial Sequence
  Synthetic Construct
MSIHMPDPDAVARMAAEGRFGIPESDLPTYHAHVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAERSSA

SEQ ID NO: 47 - protein, 513 aa, Artificial Sequence
  Synthetic Construct
MSIHMPDPDAVARMAAEGRFGIPESDLPTYHASVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSAALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAERSSA

SEQ ID NO: 48 - protein, 513 aa, Artificial Sequence
  Synthetic Construct
MSIHMPDPDAVARMAAEGRFGIPESDLPTYHASVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTQPFDVTGHPACSVPAGLADGLPVGLMIVGKHFDDATVLKVANAFEQAVGGFPTPAAERSSA

SEQ ID NO: 49 - protein, 513 aa, Artificial Sequence
  Synthetic Construct
MSIHMPDPDAVARMAAEGRFGIPESDLPTYHASVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARGLEMLANTAPFDVTGHPACSVPAGLADGLPVGLMIVGKHFDDATVLKVANAFEQAVGGFPTPAAERSSA

SEQ ID NO: 50 - protein, 513 aa, Artificial Sequence
  Synthetic Construct
MSIHMPDPDAVARMAAEGRFGIPESDLPTYHASVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTCPFDVTGHPACSVPAGLADGLPVGLMIVGKHFDDATVLKVANAFEQAVGGFPTPAAERSSA

SEQ ID NO: 51 - protein, 513 aa, Artificial Sequence
  Synthetic Construct
MSIHMPDPDAVARMAAEGRFGIPESDLPTYHASVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTKPFDVTGHPACSVPAGLADGLPVGLMIVGKHFDDATVLKVANAFEQAVGGFPTPAAERSSA

SEQ ID NO: 52 - protein, 513 aa, Artificial Sequence
  Synthetic Construct
MSIHMPDPDAVARMAAEGRFGIPESDLPTYHASVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTSPFDVTGHPACSVPAGLADGLPVGLMIVGKHFDDATVLKVANAFEQAVGGFPTPAAERSSA

SEQ ID NO: 53 - protein, 513 aa, Artificial Sequence
  Synthetic Construct
MSIHMPDPDAVARMAAEGRFGIPESDLPTYHASVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTANFDVTGHPACSVPAGLADGLPVGLMIVGKHFDDATVLKVANAFEQAVGGFPTPAAERSSA

SEQ ID NO: 54 - protein, 513 aa, Artificial sequence
  Synthetic Construct
MSIHMPDPDAVARMAAEGRFGIPESDLPTYHASVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPPAERSSA

SEQ ID NO: 55 - protein, 513 aa, Artificial sequence
  Synthetic Construct
MSIHMPDPDAVARMAAEGRFGIPESDLPTYHASVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTNPFDVTGHPACSVPAGLADGLPVGLMIVGKHFDDATVLKVANAFEQAVGGFPTPAAERSSA

SEQ ID NO: 56 - protein, 509 aa, Pseudonocardia thermophila
MIHMPDPDAVARMAAEGRFGIPVVVICRTYHDLGHRPARLVEQGRRAVRGGGPHPAAALVDPPRRRGRQARRVGGADLDHRNLGGAARRPHGRGQDNVAVAGVPMMNGSRTLEGFVPAEDATVVSRLLAAGATIAGKSVCEDLCFSGGSHTSKPGTVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVLATEGGLWQMVEGNAYGMHWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAZ